SEQUENCE LISTING (26 sequences)

SEQ ID NO: 1 - DNA, 58 bp, Homo sapiens
ccgggcgtattctctacgtggatttctcgagaaatccacgtagagaatacgctttttg

SEQ ID NO: 2 - DNA, 57 bp, Homo sapiens
ccgggcgtattctctacgtggatttctcgagaaatccacgtagagaatacgcttttt

SEQ ID NO: 3 - DNA, 58 bp, Homo sapiens
ccggagtcgctggtcccggtttatactcgagtataaaccgggaccagcgacttttttg

SEQ ID NO: 4 - DNA, 58 bp, Homo sapiens
ccggttacgattgcgacggaaatttctcgagaaatttccgtcgcaatcgtaatttttg

SEQ ID NO: 5 - DNA, 57 bp, Homo sapiens
ccggtgacagaaagagatcaggtttctcgagaaacctgatctctttctgtcattttt

SEQ ID NO: 6 - DNA, 58 bp, Homo sapiens
ccggctccctggtctccagcttaaactcgagtttaagctggagaccagggagtttttg

SEQ ID NO: 7 - DNA, 58 bp, Homo sapiens
ccgggcagttctccaatgatatctactcgagtagatatcattggagaactgctttttg

SEQ ID NO: 8 - DNA, 58 bp, Homo sapiens
ccgggggagaccatctgcgagtatactcgagtatactcgcagatggtctccctttttg

SEQ ID NO: 9 - DNA, 57 bp, Homo sapiens
ccgggcagttctccaatgatatctactcgagtagatatcattggagaactgcttttt

SEQ ID NO: 10 - DNA, 58 bp, Homo sapiens
ccggcaggcagaagagcacagatgtctcgagacatctgtgctcttctgcctgtttttg

SEQ ID NO: 11 - DNA, 57 bp, Homo sapiens
ccggcaggcagaagagcacagatgtctcgagacatctgtgctcttctgcctgttttt

SEQ ID NO: 12 - DNA, 58 bp, Homo sapiens
ccggcggccacatttatggagacaactcgagttgtctccataaatgtggccgtttttg

SEQ ID NO: 13 - DNA, 58 bp, Homo sapiens
ccggcgcaagaggtttggcatgaaactcgagtttcatgccaaacctcttgcgtttttg

SEQ ID NO: 14 - DNA, 58 bp, Homo sapiens
ccggacaatctggagcgtcagtatcctcgaggatactgacgctccagattgttttttg

SEQ ID NO: 15 - DNA, 58 bp, Homo sapiens
ccggcgtgagctattacactcgtatctcgagatacgagtgtaatagctcacgtttttg

SEQ ID NO: 16 - DNA, 62 bp, Homo sapiens
gtaccgggatggtcttaaatgctcatttctcgagaaatgagcatttaagaccatcttttttg

SEQ ID NO: 17 - DNA, 57 bp, Homo sapiens
ccggcccagtctcaataatacctatctcgagataggtattattgagactgggttttt

SEQ ID NO: 18 - DNA, 57 bp, Homo sapiens
ccggcgtcctttacccgaacaacttctcgagaagttgttcgggtaaaggacgttttt

SEQ ID NO: 19 - DNA, 57 bp, Homo sapiens
ccgggttggatacatctcgccattactcgagtaatggcgagatgtatccaacttttt

SEQ ID NO: 20 - DNA, 57 bp, Homo sapiens; identical to SEQ ID NO: 19

SEQ ID NO: 21 - DNA, 58 bp, Homo sapiens
ccggtatctccaaggcgttggtatactcgagtataccaacgccttggagatatttttg

SEQ ID NO: 22 - DNA, 58 bp, Homo sapiens
ccgggctgttcgacaagagcttgaactcgagttcaagctcttgtcgaacagctttttg

SEQ ID NO: 23 - DNA, 58 bp, Homo sapiens
ccgggccttacagacgattaactatctcgagatagttaatcgtctgtaaggctttttg

SEQ ID NO: 24 - DNA, 58 bp, Homo sapiens
ccgggctgcagatcttgtgacagaactcgagttctgtcacaagatctgcagctttttg

SEQ ID NO: 25 - DNA, 62 bp, Homo sapiens
gtaccggacgtctctctcaccaggatttctcgagaaatcctggtgagagagacgtttttttg

SEQ ID NO: 26 - DNA, 62 bp, Homo sapiens
gtaccggagagggagttgtcacgctacactcgagtgtagcgtgacaactccctctttttttg

(19) United States
(12) Patent Application Publication
(10) Pub. No.: US 2026/0069581 A1
(43) Pub. Date: Mar. 12, 2026

(54) METHODS AND COMPOSITIONS FOR CANCER THERAPY

(71) Applicant: OHIO STATE INNOVATION FOUNDATION, Columbus, OH (US)

(72) Inventors: John C. BYRD, Columbus, OH (US); Rosa LAPALOMBELLA, Columbus, OH (US); Pu ZHANG, Columbus, OH (US)

(21) Appl. No.: 18/036,825

(22) PCT Filed: Nov. 12, 2021
(86) PCT No.: PCT/US2021/059203

§ 371 Date: May 12, 2023

Related U.S. Application Data
(60) Provisional application No. 63/113,821, filed on Nov. 13, 2020.

(51) Int. Cl. A61K 31/4545; A61K 9/00; A61K 31/167; A61K 45/06; A61K 47/38

(52) U.S. Cl. CPC A61K 31/4545; A61K 9/0053; A61K 31/167; A61K 45/06; A61K 47/38

(57) ABSTRACT

In one aspect, the disclosure relates to relates to methods of treatment of AML and pharmaceutical compositions for treatment of same. The methods of the present disclosure for the treatment of AML comprise the step of administering to the mammal a therapeutically effective amount of: (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor, or a pharmaceutically acceptable salt, hydrate, solvate, or polymorph thereof. This abstract is intended as a scanning tool for purposes of searching in the particular art and is not intended to be limiting of the present disclosure.

### CROSS-REFERENCE TO RELATED APPLICATIONS

This Application claims the benefit of U.S. Provisional Application No. 63/113,821, filed on Nov. 13, 2020, which is incorporated herein by reference in its entirety.

### STATEMENT REGARDING FEDERALLY SPONSORED RESEARCH

This disclosure was made with U.S. Government support under grant numbers R35 CA197734 and R01 CA223165, awarded by the National Institute of Health (National Cancer Institute—Center for Cancer Research). The U.S. government has certain rights in the disclosure.

### REFERENCE TO A SEQUENCE LISTING SUBMITTED AS A TEXT FILE VIA EFS-WEB

The Sequence Listing submitted Nov. 12, 2021 as a text file named “OSIF00006U_USPRV01.txt,” created on Nov. 13, 2020, and having a size of 4,304 bytes, is hereby incorporated by reference pursuant to 37 C.F.R. § 1.52(e)(5).

### BACKGROUND

Acute myeloid leukemia (AML) is the most common diagnosed leukemia in adults, with ˜19,940 cases expected in 2020 and a median age at diagnosis of 68 years (Refs. 1 and 2). The overall prognosis of the disease remains poor, with median 5-year survival of less than 10% for patients over 60 years old. While several therapies have recently been approved to treat different subtypes of AML, only modest benefit has been reported.

Despite advances in cancer research, there is still a scarcity of clinically effective treatments for AML. These needs and other needs are satisfied by the present disclosure.

### SUMMARY

In accordance with the purpose(s) of the invention, as embodied and broadly described herein, the invention, in one aspect, relates to methods of treatment of AML and pharmaceutical compositions for treatment of same.

Disclosed herein are pharmaceutical compositions comprising at least one NAMPT inhibitor, at least one HDAC8 inhibitor and/or at least one SIRT6 inhibitor. In a further aspect, the pharmaceutical compositions comprise at least one NAMPT inhibitor and at least one HDAC8 inhibitor. In a still further aspect, the pharmaceutical compositions comprise at least one NAMPT inhibitor and at least one SIRT6 inhibitor.

In a yet further aspect, the disclosed pharmaceutical compositions can comprise a first dosage form comprising at least one NAMPT inhibitor, and a second dosage form comprising at least one HDAC8 inhibitor and/or at least one SIRT6 inhibitor. The first and second dosage forms can be co-administered dosage forms, e.g., sequentially or simultaneously co-administered dosage forms such that a first dosage form comprises at least one disclosed NAMPT inhibitor, or a pharmaceutically acceptable salt, hydrate, solvate, or polymorph thereof; and a second dosage form comprises at least one HDAC8 inhibitor and/or at least one SIRT6 inhibitor. In various aspects, the pharmaceutical composition comprises: (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor, or a pharmaceutically acceptable salt, hydrate, solvate, or polymorph thereof. In a still further aspect, the cancer is AML. In various aspects, the pharmaceutical composition comprising

Also disclosed are methods for the treatment of AML comprising the step of administering to the mammal a therapeutically effective amount of: (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor, or a pharmaceutically acceptable salt, hydrate, solvate, or polymorph thereof. In a still further aspect, the cancer is AML. at least one disclosed compound or pharmaceutically acceptable salt thereof. In a further aspect, the administering is co-administering. In a still further aspect, the co-administering is sequential administration of: (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor, or a pharmaceutically acceptable salt, hydrate, solvate, or polymorph thereof. In a still further aspect, the cancer is AML. In a yet further aspect, the co-administering is simultaneous administration of: (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor, or a pharmaceutically acceptable salt, hydrate, solvate, or polymorph thereof. In a still further aspect, the cancer is AML. In an even further aspect, simultaneous administration can comprise administration of a first dosage form comprising at least one disclosed NAMPT inhibitor, or a pharmaceutically acceptable salt, hydrate, solvate, or polymorph thereof; and of a second dosage form comprising at least one HDAC8 inhibitor and/or at least one SIRT6 inhibitor. In a still further aspect, the cancer is AML. In a still further aspect, simultaneous administration can comprise administering a fixed dose combination dosage form comprising: (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor, or a pharmaceutically acceptable salt, hydrate, solvate, or polymorph thereof. In a still further aspect, the cancer is AML.

Also disclosed are methods for inhibit NAMPT activity and HDAC8 activity or SIRT6 activity in at least one cell, comprising the step of contacting the cell with an effective amount of: (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor, or a pharmaceutically acceptable salt, hydrate, solvate, or polymorph thereof. In a still further aspect, the cancer is AML.

Also disclosed are uses of a disclosed pharmaceutical composition comprising: (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor, or a pharmaceutically acceptable salt, hydrate, solvate, or polymorph thereof. In a still further aspect, the cancer is AML.

Also disclosed are uses of a disclosed compound, or a pharmaceutically acceptable salt thereof, in the manufacture of a medicament for the treatment of AML, wherein the medicament comprises: (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor, or a pharmaceutically acceptable salt, hydrate, solvate, or polymorph thereof. In a still further aspect, the cancer is AML.

Also disclosed are kits comprising at least one disclosed NAMPT inhibitor, or a pharmaceutically acceptable salt, hydrate, solvate, or polymorph thereof, and at least one HDAC8 inhibitor and/or at least one SIRT6 inhibitor, or a pharmaceutically acceptable salt, hydrate, solvate, or polymorph thereof, and and one or more of: (a) at least one agent known to treat a disorder associated with uncontrolled cellular proliferation activity; or (b) instructions for treating AML.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1C show representative data for genome-wide functional screening identifies two histone deacetylase genes as combination targets for NAMPT inhibitor. FIG. 1A shows a schematic illustration of CRISPR screen workflow. The CRISPR screen was conducted by transfecting GeCKO library into MOLM13 cells. Virus MOI was titrated to ensure a single sgRNA to be transduced into each cell. 50 nM KPT-9274 or DMSO vehicle was administrated to CRISPR library-transfected MOLM13 cells after puromycin selection. FIG. 1B shows representative genes ranked by their −log(p-values) and displayed against each gene's corresponding ranks of essentiality. Hits for validation are highlighted. p-values of genes were calculated based on sgRNA efficiency. FIG. 1C shows representative data for sgRNAs targeting selected hits are constantly depleted following KPT-9274 treatment.

FIGS. 2A-2M show show representative data for genetic depletion of HDAC8 or SIRT6 increases vulnerability of AML cell lines to KPT-9274 treatment. FIGS. 2A-2D show data for knockout efficiency of CDS- and 3′UTR-targeting shRNAs in MOLM13 and Kasumi-1 cells as assessed with Western blotting (FIGS. 2A, 2D) with GAPDH as a loading control. Densitometric quantification of protein band intensity is shown (n=2) in FIGS. 2B, 2E. FIGS. 2E-2H show representative data for depletion of HDAC8 or SIRT6 (as indicated in graphs) and sensitization of AML cell lines to KPT-9274 treatment. Dose-response curves of shRNA-stable MOLM13 and Kasumi-1 cells for 24-hour and 48-hour treatments are plotted. Cell viability was measured with MTS. Results are shown as mean±SEM of 4 technical replicates and 3-4 biological replicates. *p<0.05;**p<0.01. p-values for 24 hour KPT-9274 treatment on Kasumi-1 were not determined, as drug treatment did not achieve IC₅₀ on scrambled control. FIGS. 2I-2J show representative for depletion of HDAC8 or SIRT6 and that shRNA synergizes with KPT-9274 to enhance apoptosis of AML cell lines. Scrambled or gene-targeting shRNA-stable MOLM13 or Kasumi-1 cells were treated with IC₂₀ dose of KPT-9274 for 48 hour and stained with Annexin/PI for flow cytometry analysis. Data are shown as mean±SEM of % population from duplicates. FIGS. 2K-2L show representative data that KPT-9274 attenuates AML self-renewal and induces myeloid differentiation in HDAC8 or SIRT6-depleted cells. Quantification of the number of colonies formed by shRNA-stable Kasumi-1 and MOLM13 cells in the presence or absence of KPT-9274 in CFU assays. Indicated number of cells were seeded into Methocult and colonies were counted and replated by a double-blind approach after 7 days. Results are shown as mean±SEM of duplicates. *p<0.05. **p<0.01. FIG. 2M shows representative images of colony morphologies and Giemsa staining of cytospin preparations (as indicated for rows) of cells derived from KPT-9274-treated MOLM13 CFU with indicated nucleic acid used for depletion. For each condition, images were randomly taken from 6-12 fields of view. Scale bar=50 μm.

FIGS. 3A-3D show representative obtained using a combination of AR-42 (which is a a pan-HDAC inhibitor) and KPT-9274 showing that the combination synergistically reduces survival and self-renewal of AML cells. FIG. 3A shows representative data for drug synergy matrix plots for the combination of AR-42 and KPT-9274 in AML cell lines, MOLM13 and Kasumi-1. MOLM13 and Kasumi-1 cells were treated with a range of dosages of AR-42 and KPT-9274 for 48 hours and 72 hours. Dose response curves and EC₅₀s for single agents are shown. n=2. FIG. 3B shows representative synergy score calculations from data obtained using IDH2-mutant and IDH2-wildtype (WT) TF-1 erythroleukemia cell lines. FIG. 3C shows representative synergy score calculations from data obtained using IDH1-mutant and -WT patient cells. The data used to calculate the synergy scores were obtained by treating cells with pairwise combinations of AR-42 and KPT-9274. After treatment, MTS was added to the plate to assess mitochondria metabolism. Synergy scores were calculated with Combenefit based on HSA model. FIG. 3D shows data for quantification of CFU colonies formed by CD34⁺ healthy donor bone marrow cells and AML patient cells which were treated with vehicle, 0.1 μM KPT-9274, 0.8 μM AR-42 and drug combination.

FIGS. 4A-4D show representative data that simultaneous inhibition of HDAC8 and NAMPT exhibits enhanced in vivo therapeutic efficacy in a mouse xenograft model of human AML. FIG. 4A shows a schematic illustration of experimental design. NCG mice were engrafted with luciferase-tagged MOLM13 cells and treated with vehicle, 20 mg/kg AR-42, 100 mg/kg KPT-9274 and AR-42/KPT-9274 in combination. FIG. 4B shows representative IVIS imaging displays the changes of luciferase signals over time as indicated for different treatments as indicated. An enlargement of the relative values corresponding to colors in the image is shown to the left. FIG. 4C shows representative histopathology of bone marrow and liver sections as indicated from each treatment group as indicated. Black box shows the evidence of differentiated hematopoietic cells. FIG. 4D shows Kaplan-Meier analysis of the mouse survival (Cox proportional hazards model; KPT-9274 vs combination p<0.005; AR-42 vs combination p<0.02; vehicle vs combination p<0.001). n=8-9 mice/group.

FIG. 5A-5E show representative data that AR-42 and KPT-9274 synergistically alter DNA repair gene sets of LIC transcriptomes. That is, the data show that combination therapy changes the transcriptomes of patient leukemia initiating cells (LICs). Briefly, primary cells from 5 patients were treated with drug combination or vehicle for 12 hours before LICs (CD34⁺ CD38⁻) were sorted for LC-RNA-seq. FIG. 5A shows a representative PCA plot showing that drug combination-treated LICs tend to form distinct clusters from vehicle-treated LICs. FIG. 5B shows a representative Volcano plot for significantly upregulated and downregulated DEGs in drug combination-treated LICs. Cutoff Log 2FC=1.5; p-value<10e⁻². FIG. 5C shows representative heatmaps showing the relative expressions of genes (normalized read counts) within DNA damage repair gene sets of LICs as being treated with indicated drug(s) relative to vehicle controls in 5 patients. The hierarchical clustering of genes and samples was performed using Euclidean distance matrix and Ward's clustering method. FIG. 5D shows representative normalized read count data of selected DNA repair genes in LICs across patients under different treatment conditions. FIG. 5E shows representative IPA analysis of enriched pathways of differentially expressed genes in drug combination-treated LICs.

FIGS. 6A-6G show representative data for HDAC8 inhibition and SIRT6 knockdown demonstrating sensitization of AML to KPT-9274 by suppression of HR and D-NHEJ pathways and mono-ADP-ribosylation of PARP1. FIG. 6A shows representative data that combined treatment of AR-42 and KPT-9274 synergistically increases unrepaired DNA damage sites as indicated by the accumulation of phosphorylated H2A.X. MOLM13, IDH2-WT TF-1 and IDH2-mutant (MUT) TF-1 cells were incubated with vehicle control, 0.8 μM AR-42 alone, 0.1 μM KPT-2974 alone and drug combination for 48 hours. Thereafter, cells were fixed, permeabilized and stained with BV421 mouse anti-H2A.X (pS139) (Clone N1-431). Stained cells were analyzed by flow cytometry. Results are shown as mean±SEM of duplicates. *p<0.05. N.S.: not significant. FIG. 6B (Left panel) shows a schematic illustration of I-SceI-based EJDR reporter system; and FIG. 6B (Right panel) shows representative data that AR-42 blocks HR and NHEJ repairs, while KPT-9274 blocks NHEJ repair. Results are shown as mean±SEM of duplicates. *p<0.05. FIG. 6C shows representative data that exposure of MOLM13 cells to AR-42 and KPT-9274 results in a decrease in activities of HR, NHEJ and ATM signaling, and a concomitant increase in phospho-H2A.X (pS139) (Clone 20E3), consistent with the loss of multiple pathway-mediated DSB repair. MOLM13 cells were treated with vehicle control, 0.4 μM or 0.8 μM AR-42 alone, 0.1 μM or 0.25 μM KPT-2974 alone and drug combination for 24 hours before being subject to Western blotting analysis of HR, NHEJ and ATM markers. GAPDH served as loading control. Results are representative of two independent experiments. In the FIG. the treatment concentrations, protein detected in the given Western blot, and molecular weight of the protein detected are as shown in the figure. FIG. 6D shows representative data for knockdown of HDAC8 reducing the levels of HR pathway mediators and abolishes the activities of D-NHEJ and ATM signaling in cooperation with KPT-9274. Scrambled or shHDAC8-transduced MOLM13 cells were treated with vehicle, 0.1 μM or 0.25 μM KPT-2974 before being subject to immunoblotting analysis of DNA repair targets. GAPDH serves as loading control. Results are representative of two independent experiments. In the FIG. the treatment concentrations, protein detected in the given Western blot, and molecular weight of the protein detected are as shown in the figure. FIG. 6E shows representative data for shSIRT6 and KPT-9274 synergistically increasing unrepaired DNA damage sites as indicated by the accumulation of phosphorylated H2A.X. Results are shown as mean±SEM of duplicates. **p<0.01. FIG. 6F shows representative data from a co-IP experiment showing that shSIRT6 decreases mono-ADP-ribosylation of PAPR1 in response to KPT-9274 treatment. PAPR1 was immunoprecipitated and mono-ADP-ribosylation was detected by western blotting. Results are representative of two independent experiments. FIG. 6G shows a schematic illustration of possible mechanisms of sensitizing AML cells to NAMPT inhibition by suppression of HDAC8 or SIRT6.

FIGS. 7A-7B show representative data for knockdown of selected genes has modest effects on MOLM13 responses to KPT-9274 treatment. FIG. 7A shows data pertaining to hits from shRNA and CRISPR knockdown screening (concentration shown on x-axis). FIG. 7B shows data pertaining to various HDAC isoforms that were knocked down by shRNA or CRISPR. For the graphs in FIGS. 7A-7B, the responses of parental, shRNA-stable and sgRNA-transfected MOLM13 cells to KPT-9274 treatment (48-hour treatments) are plotted. Cell viability was measured with MTS (y-axis). Results are shown as mean±SEM of 4 technical replicates and 3 biological replicates. Western blotting for knockdown efficiency is shown.

FIGS. 8A-8C show representative data pertaining to the effect of combined treatment of KPT-9274 and an HDAC8-specific inhibitor (PCI-34051) showing synergistic effects on AML cell lines. FIG. 8A shows data obtained in Kasumi-1 cells. FIG. 8B shows data obtained in MOLM13 cells. FIG. 8C shows data obtained in MV4-11 cells. In each of FIGS. 8A-8C, the indicated cells were treated with different dosages of PCI-34051 for 24 hours before receiving KPT-9274 for 48 hours. MTS was added to the plate to assess mitochondria metabolism. The results were analyzed with Combenefit to quantify synergy levels of HDAC8i/NAMPTi pairwise combinations. The synergy scores were calculated based on the HSA model and shown in matrices. n=1.

FIGS. 9A-9B show representative data that AR-42 treatment sensitizes MV4-11 and OCI-AML3 cell lines to KPT-9274 treatment. FIG. 9A shows obtained in MV4-11 cells, whereas FIG. 9B shows data obtained in OCI-AML3 cells. In each of FIGS. 9A-9B, the cells were treated with pairwise combinations of doses of AR-42 and KPT-9274 for 48 hours. MTS was added to the plate to assess mitochondria metabolism. The results were analyzed with Combenefit to quantify synergy levels. The synergy scores were calculated based on the HSA model and shown in matrices. n=2.

FIGS. 10A-10D show representative data that the combined treatment of AR-42 and KPT-9274 leads to mitochondrial potential collapses in cell lines. FIG. 10A shows data obtained in MOLM13 cells. FIG. 10B shows data obtained in Kasumi-1 cells. FIG. 10C shows data obtained in MV4-11 cells. In the experiment, cells were treated with vehicle control, 0.8 μM AR-42 alone, 0.1 μM KPT-2974 alone or drug combo for indicated periods of time before being stained by TMRM in combination with Annexin V-FITC followed by flow cytometric analysis. n=2. FIG. 10D shows data obtained in MOLM-13 cells that were treated with the indicated concentrations of AR-42 and/or KPT-9274. The survival of cells was analyzed by annexin V/PI labeling. *P<0.05, ***P<0.001, ****P<0.0001 vs vehicle control; \hat{ }\hat{ }\hat{ }\hat{ }P<0.0001 vs AR-42 treatment alone.

FIGS. 11A-11B show representative data that combinations of AR-42 and KPT-9274 have no overlapping toxicities in vivo using MOLM13 xenograft mice. FIG. 11A show H&E images of tissues obtained from MOLM13 xenograft mice that with the magnification and treatments as indicated in the figure. The images were representative of 3 different fields. The data show that mice in the combination treatment group displayed atrophy of seminiferous tubules and decreased to absent spermatogenensis. FIG. 11B show weight change data of MOLM13 xenograft mice in response to different treatments.

FIG. 12 shows representative data for normalized read counts (y-axis) of selected DNA repair genes in LIC RNA-seq across patients under different treatment conditions as indicated in each graph.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Thus, for example, reference to “a patient,” “a cancer,” or “a pharmaceutical composition,” including, but not limited to, two or more such patients, cancers, or pharmaceutical compositions.

Reference to “a/an” chemical compound, protein, and antibody each refers to one or more molecules of the chemical compound, protein, and antibody rather than being limited to a single molecule of the chemical compound, protein, and antibody. Furthermore, the one or more molecules may or may not be identical, so long as they fall under the category of the chemical compound, protein, and antibody. Thus, for example, “an” antibody is interpreted to include one or more antibody molecules of the antibody, where the antibody molecules may or may not be identical (e.g., different isotypes and/or different antigen binding sites as may be found in a polyclonal antibody).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “inhibit” or other forms of the word such as “inhibiting” or “inhibition” means to hinder or restrain a particular characteristic. It is understood that this is typically in relation to some standard or expected value, i.e., it is relative, but that it is not always necessary for the standard or relative value to be referred to. For example, “inhibits” means hindering, interfering with or restraining the activity of the gene relative to a standard or a control. “Inhibits” can also mean to hinder or restrain the synthesis, expression or function of the protein relative to a standard or control.

As used herein, the terms “inhibit” and “inhibition” mean to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, “NAMPT” refers to nicotinamide phosphoribosyltransferase.

As used herein, “NAMPTi” or “NAMPT inhibitor”, which may be used interchangeably, refers to a compound or pharmaceutical composition that decreases the activity of nicotinamide phosphoribosyltransferase. The mechanism of decreasing activity can be as a competitive inhibitor, non-competitive inhibitor, partial allosteric modulator, irreversible inhibitor, and any other mechanism of decreasing the activity of nicotinamide phosphoribosyltransferase.

As used herein, “HDAC8” refers to the NAD⁺-dependent histone deacetylase isoform known as histone deacetylase 8, which has also been refererred to as the protein encoded by the gene referred to as histone deacetylase 8, histone deacetylase-like 1, HDACL1, KDAC8, RPD3, HD8, EC 3.5.1.98, WTS, CSLS5, and CDA07. These names may also refer to the protein itself. HDAC8 is responsible for the deacetylation of lysine residues on the N-terminal part of the core histones (H2A, H2B, H3 and H4). Histone deacetylation gives a tag for epigenetic repression and plays an important role in transcriptional regulation, cell cycle progression and developmental events. Enzymatically, histone deacetylases act via the formation of large multiprotein complexes. Also involved in the deacetylation of cohesin complex protein SMC3 regulating release of cohesin complexes from chromatin. May play a role in smooth muscle cell contractility.

As used herein, “HDAC8i” or “HDAC8 inhibitor”, which may be used interchangeably, refers to a compound or pharmaceutical composition that decreases the activity of histone deacetylase 8. The mechanism of decreasing activity can be as a competitive inhibitor, non-competitive inhibitor, partial allosteric modulator, irreversible inhibitor, and any other mechanism of decreasing the activity of histone deacetylase 8.

As used herein, “SIRT6” refers to the NAD⁺-dependent histone deacetylase isoform known as NAD-dependent protein deacetylase sirtuin-6. SIRT6 can also refer to the gene encoding the protein SIRT6. SIRT6 has deacetylase activity towards histone H3K9Ac and H3K56Ac; and modulates acetylation of histone H3 in telomeric chromatin during the S-phase of the cell cycle. SIRT6 deacetylates histone H3K9Ac at NF-kappa-B target promoters and may down-regulate the expression of a subset of NF-kappa-B target genes and acts as a corepressor of the transcription factor HIF1A to control the expression of multiple glycolytic genes to regulate glucose homeostasis. SIRT6 is required for genomic stability and regulates the production of TNF protein. It is believed that SIRT6 has a role in the regulation of life span (by similarity). Deacetylation of nucleosomes interferes with RELA binding to target DNA. SIRT6 may be required for the association of WRN with telomeres during S-phase and for normal telomere maintenance. It is believed that SIRT6 is required for genomic stability. It promotes DNA end resection via deacetylation of RBBP8.

As used herein, “SIRT6i” or “SIRT6 inhibitor” refers to a compound or pharmaceutical composition that decreases the activity of NAD-dependent protein deacetylase sirtuin-6. The mechanism of decreasing activity can be as a competitive inhibitor, non-competitive inhibitor, partial allosteric modulator, irreversible inhibitor, and any other mechanism of decreasing the activity of NAD-dependent protein deacetylase sirtuin-6.

As used herein, “AR-42” refers to (S)—N-Hydroxy-4-(3-methyl-2-phenylbutanamido)benzamide and is associated with CAS No. 935881-37-1. It is also referred to as AR42, OSU-HDAC42, AR 42, and (S)-HDAC-42. AR-42 has a structure represented by the chemical formula:

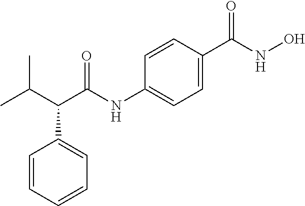 [SMILES: CC(C)[C@H](C(=O)Nc1ccc(C(=O)NO)cc1)c1ccccc1]

As used herein, “KPT-9274” refers to (E)-3-(6-aminopyridin-3-yl)-N-[[5-[4-(4,4-difluoropiperidine-1-carbonyl)phenyl]-7-(4-fluorophenyl)-1-benzofuran-2-yl]methyl]prop-2-enamide and is associated with CAS No. 1643913-93-2. It is also referred to as KPT9274, PAK4-IN-1, PAK4-IN-1KPT9274, and UNII-9T56TV18X7. KPT-9274 has a structure represented by the chemical formula:

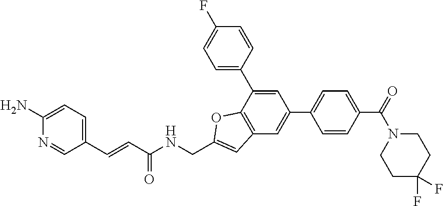 [SMILES: Nc1ccc(/C=C/C(=O)NCc2cc3cc(-c4ccc(C(=O)N5CCC(F)(F)CC5)cc4)cc(-c4ccc(F)cc4)c3o2)cn1]

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and juvenile subjects, whether male or female, are intended to be covered. In one aspect, the subject is a mammal. A patient refers to a subject afflicted with a disease or disorder. The term “patient” includes human and veterinary subjects.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as AML. The effect can be therapeutic in terms of a partial or complete cure of a disease, condition, symptom or adverse effect attributed to the disease, disorder, or condition. The term “treatment” as used herein can include any treatment of AML in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### B. INTRODUCTION

KPT-9274 is a phase I NAMPT inhibitor which induces accumulation of DNA breaks by depleting NAD⁺ supply. In the present disclosure, exemplary data shows data obtained from an unbiased CRISPR screen against AML which identified two histone deacetylase members, HDAC8 and SIRT6, and validated HDAC8 pharmacologically with AR-42 and SIRT6 genetically with shRNA as synthetic lethal targets regulating KPT-9274 sensitivity. The data in the present disclosure demonstrate that targeting DNA repair functions of HDAC8 or SIRT6 can be provide a therapeutic strategy sensitizing leukemia initiating cells (LICs) to KPT-9274. Moreover, the data in the present disclosure support preclinical investigation of SIRT6 as a lethal target in NAMPT-inhibited leukemia and the development of potent therapeutic agents that target its mono-ADP-ribosylase activity while avoiding deleterious effect on its normal function. As AR-42 has been investigated in phase I trials, the present disclosure further demonstrates a basis for a rational and clinically testable combination therapy with KPT-9274 at less toxic doses to treat patients with AML.

AML cells are addicted to nicotinamide phosphoribosyltransferase (NAMPT)-mediated salvage pathway for NAD⁺ biosynthesis (Refs. 3 and 4). NAD⁺ functions as a co-enzyme for redox reactions in numerous metabolic pathways and serves as a substrate for poly (ADP-ribose) polymerase (PARP), regulating DNA damage repair (DDR) gene expression and stress responses. Thus, targeting the NAMPT-dependent NAD⁺ generation has gained attention as a potential therapeutic strategy in AML and NAMPT inhibitors have moved to phase I trials. In previous studies, the preclinical efficacy was demonstrated for targeting NAMPT on eliminating AML in vitro and in vivo by employing a potent NAMPT inhibitor, KPT-9274 (Ref. 5). Although KPT-9274 has shown activity towards PAK4 in other cancers, it was found that overexpression or knockdown of PAK4 did not affect the proliferation of AML cell lines nor the activity of KPT-9274 (Ref. 5). Therefore, prior data suggested that the anti-AML property of KPT-9274 was solely dependent on inhibiting NAMPT rather than PAK4.

Although the pre-clinical data are compelling, the present disclosure posits that NAMPT inhibitors will likely be ineffective as monotherapies based upon AML metabolic plasticity ultimately permitting resistance. For example, in some tumors, as a result of enhancer remodeling, acquired resistance to NAMPT inhibitors has been observed via nicotinamide riboside kinase 1 (NMRK1)-dependent synthesis of NAD⁺ (Ref. 6). Composition of gut bacteria has also been shown to confer host resistance to NAMPT inhibition by engaging the deamidated biosynthesis pathways (Ref. 7). In addition, the anti-leukemic activity of NAMPT inhibitors is dependent on functional p53 whose expression is tumor subtype-specific (Ref. 8). Lastly, dose-limiting toxicities pose another barrier for clinical success of the inhibitor (Refs. 9-10). In clinical trials with the 1^{st} generation NAMPT inhibitor (NAMPTi), dose-limiting toxicities were observed, such as thrombocytopenia and gastrointestinal, retinal and cardiac toxicities (Ref. 11). Unlike 1^{st} generation NAMPTi, KPT-9274 was shown to be better tolerated in Phase I trial on solid tumor and non-hodgkins lymphoma. However, drug-related adverse events including anemia and fatigue were reported. Therefore, strengthening the clinical efficacy of NAMPTis through rational combination therapies represents an unmet need in AML. It is necessary to develop strategies that allow lowering the doses of NAMPT is to improve the therapeutic window and also to prevent drug resistance.

To address this, the studies disclosed herein utilized an unbiased genome-wide CRISPR screen to identify genes that upon depletion confer the sensitivity to KPT-9274 in AML cells. Among the hits, the studies disclosed herein identify two genes encoding NAD⁺-dependent histone deacetylases, namely HDAC8 and SIRT6, as being of interest for new therapeutic approaches lowering does of NAMPT inhibitors useful for treating AML. Without wishing to be bound by a particular theory, it is believed that modulation of SIRT6 may be useful in this regard because SIRT6 participates in DDR by deacetylating and activating DNA-dependent protein kinase, catalytic subunit (DNA-PKcs) and CtIP in non-homologous end joining (NHEJ) and homologous recombination (HR) pathways (Refs. 12-15). In addition, without wishing to be bound by a particular theory, it is believed that modulation of SIRT6 may be useful in this regard because SIRT6 activates poly(ADP-ribose) polymerase 1 (PARP1) by mono-ADP-ribosylating PARP1 to repair DNA breaks (Ref. 13). Without wishing to be bound by a particular theory, it is believed that modulation of HDAC may be useful in this regard because HDAC8, a unique class I HDAC, has been shown to be recruited to DNA double strand break (DSB) sites with Rad51 and Smc3, members of the HR pathway in multiple myeloma (Ref. 16). Moreover, without wishing to be bound by a particular theory, it is believed that modulation of HDAC may be useful in this regard because HDAC8 inhibition increases DNA damage (Ref. 17). Without wishing to be bound by a particular theory, it is believed that the modulation of HDAC may be useful in this regard because in AML, tyrosine kinase inhibitor treatment upregulated HDAC8 expression which promotes drug resistance and leukemia maintenance (Ref. 18).

It is known that AML cells accumulate high degrees of spontaneous and chemotherapy-induced DNA lesions, involving lethal DSBs. Targeting DNA repair processes can be a potential therapeutic approach to eliminate AML cells by accumulating detrimental DSBs. DSBs are primarily repaired by two processes: BRCA1/2-mediated HR and DNA-PKcs-NHEJ (D-NHEJ). PARP1 also plays critical roles in repairing lethal DSBs by facilitating the back-up NHEJ (B-NHEJ; Ref. 19). NAMPT inhibition suppresses the production of NAD⁺ to compromise the function of downstream PARP1-mediated B-NHEJ repairs. Therefore, without wishing to be bound by a particular theory, it is believed that concurrent inhibition of NAMPT and factors involved in compensatory DDR pathways may achieve the goal of improving the therapeutic index and effectiveness of NAMPTis in AML.

Disclosed herein in the examples below, it was determined that the efficacy of combined pharmacologic inhibition of NAMPT with genetic or pharmacologic inhibition of HDAC8 or SIRT6 in AML cell lines and primary patient samples, and investigated the mechanisms that may contribute to the effect. Without wishing to be bound by a particular theory, it is believed that that HDAC8i or SIRT6i-mediated deficiencies in pathways for DSB repair sensitize AML cells to synthetic lethality orchestrated by NAMPTi.

### C. COMPOUNDS

Compounds useful in the disclosed pharmaceutical compositions comprise one or more NAMPT inhibitor in combination with one or more HDAC inhibitor, e.g., an HDAC8 inhibitors and/or one or more SIRT6 inhibitor.

Suitable NAMPT inhibitors include those disclosed in U.S. Patent Publ. No. 2016/0367541; U.S. Pat. Nos. 5,696,140, 5,563,160, 6,525,077, 7,253,193 and 6,255,323; and International Patent Application Publications WO 1998/54141, WO 1998/54143, WO 1998/54144, WO 1998/54145, WO 2000/61559, WO 2000/61561, WO 1994/006770, WO 2003/097602, WO 2009/086835, WO 2009/156421, WO 2010/023307, WO 2010/066709, WO 2009/074749, WO 2010/004198, WO 1997/048696 and WO 2000/061561. However, it is believed that the compounds disclosed in International Patent Application No. PCT/US2011/026752, filed Mar. 1, 2011, and published as WO 2011/109441 are well suited for use in the disclosed pharmaceutical compositions and therapeutic methods. The compounds of the foregoing are incorporated herein by reference.

In a further aspect, the NAMPT inhibitor is selected among the group consisting of FK866, CHS-828, GNE-617, GNE-618, and KPT-9274. FK966 is also known by the chemical name 2-(E)-N-[4-(1-Benzoyl-4-piperidinyl)butyl]-3-(3-pyridinyl)-2-propenamide hydrochloride.

In a further aspect, the NAMPT inhibitor can include FK866 ((E)-N-[4-(1-benzoyl-4-yl)-butyl]-3-(pyridin-3-yl) acrylamide), CHS-828 (N-[6-(4-chlorophenoxy)hexyl]-N′-cyano-N″-4-pyridinyl-guanidine (also referred to as GMX1778), GNE-617 (N-(4-((3,5-difluorophenyl)sulfonyl)-benzyl)imidazo[1,2-a]pyridine-6-carboxamide), GNE-618 (N-[[4-[[3-(Trifluoromethyl)phenyl]sulfonyl]phenyl]methyl]-1H-pyrazolo[3,4-b]pyridine-5-carboxamide), STF118804 (4-[5-Methyl-4-[[(4-methyl-phenyl)sulfonyl]methyl]-2-oxazolyl]-N-(3-pyridinylmethyl)benzamide), KPT-9274 ((E)-3-(6-amino-pyridin-3-yl)-N-[[5-[4-(4 4-difluoropiperidine-1-carbonyl)phenyl]-7-(4-fluorophenyl)-1-benzofuran-2-yl]methyi]prop-2-enamide), and/or LSN3154567 (2-hydroxy-2-methyi-N-[1,2,3,4-tetrahydro-2-[2-(3-pyridinyloxy)acety!]-6-isoquino!inyl]-1-propane-sulfonamide.

For more information regarding GNE-618 and GNE-617, see WO2017162840; Olesen et al., Biochem Biophys Res Commun 2008; 367:799-804; and Beauparlant et al., Anticancer Drugs. 2009 June. 20(5): 346-54). For more information regarding STF118804, see Sampath et al., Pharmacology & Therapeutics (2014), http://dx.doi.Org/10.1016/j.pharmthera.2015.02.004. For more information regarding KPT-9274, see Rane et al., Nature Scientific Reports, 7:42555 (DOI: 10.1038/srep42555; Karyopharm Therapeutics; under Phase I development for the treatment of advanced solid malignancies including sarcoma, colon cancer, lung cancer, triple negative breast cancer, renal cell carcinoma, acute myeloid leukemia and non-Hodgkin's lymphoma). For more information regarding LSN3154567, see Zhao et al., DOI: 10.1158/1535-7163. MCT-16-0674).

Suitable HDAC8 inhibitors include those disclosed in U.S. Pat. Publ. Nos. 2011/0081409, 2014/0004174, 2015/0320758, 2013/0156727, 2015/0352079, 2015/0299119; U.S. Pat. Nos. 8,466,193, 8,900,565, 8,338,416, 10087149, 8906954, 9371281, 9745253, 10266487, 10508077, 10308596, 10723705, and 7820711; and International Patent Application Publication WO/2015/026935. The compounds of the foregoing are incorporated herein by reference.

In a further aspect, the HDAC inhibitor is AR-42 or PCI-2481.

Suitable SIRT6 inhibitors include those disclosed in International Patent Application Publication WO/2014/170875.

In various aspects, it is contemplated herein that the disclosed compounds further comprise their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, it is contemplated herein that the disclosed compounds further comprise their isotopically-labelled or isotopically-substituted variants, i.e., compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1-methylene-bis-(2-hydroxy-3-naphthoate)) salts. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### D. PHARMACEUTICAL COMPOSITIONS

In various aspects, the present disclosure relates to pharmaceutical compositions comprising a therapeutically effective amount of at least one NAMPT inhibitor, or a pharmaceutically acceptable salt thereof, and/or at least one of (a) at least one HDAC8 inhibitor, a pharmaceutically acceptable salt thereof; and/or (b) at least one SIRT6 inhibitor, or a pharmaceutically acceptable salt thereof.

In a further aspect, the pharmaceutical compositions can comprise a first dosage form of at least one NAMPT inhibitor, or a pharmaceutically acceptable salt thereof and a second dosage form of (a) at least one HDAC8 inhibitor, a pharmaceutically acceptable salt thereof; and/or (b) at least one SIRT6 inhibitor, or a pharmaceutically acceptable salt thereof, such the the first dosage form and the second dosage form can be used simultaneously or sequentially in a disclosed method of treatment.

In a further aspect, the pharmaceutical composition can comprise a fixed dose combination dosage form comprising at least one NAMPT inhibitor, or a pharmaceutically acceptable salt thereof, and at least one of (a) at least one HDAC8 inhibitor, a pharmaceutically acceptable salt thereof; and/or (b) at least one SIRT6 inhibitor, or a pharmaceutically acceptable salt thereof.

As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one NAMPT inhibitor, or a pharmaceutically acceptable salt thereof, and/or at least one of (a) at least one HDAC8 inhibitor, a pharmaceutically acceptable salt thereof; and/or (b) at least one SIRT6 inhibitor, or a pharmaceutically acceptable salt thereof as an active ingredient(s), a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with an basoc reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require of inhibition of NAMPT activity and/or inhibition of HDAC8 activity and/or inhibition of SIRT6 activity, an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for inhibition of NAMPT activity and/or inhibition of HDAC8 activity and/or inhibition of SIRT6 activity (e.g., treatment of AML) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of a disclosed compound for inhibition of NAMPT activity and/or inhibition of HDAC8 activity and/or inhibition of SIRT6 activity and a further therapeutic agent, e.g., an anticancer therapeutic agent. The present disclosure also relates to such a combination for use as a medicine. The present disclosure also relates to a product comprising (a) a disclosed compound for inhibition of NAMPT activity and/or inhibition of HDAC8 activity and/or inhibition of SIRT6 activity, a pharmaceutically acceptable salt, a hydrate thereof, a solvate thereof, a polymorph thereof, and (b) an additional anticancer therapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

### E. METHODS OF TREATING AML

In a further aspect, the present disclosure provides methods of treatment comprising administration of a therapeutically effective amount of a disclosed compound or pharmaceutical composition as disclosed herein above to a subject in need thereof.

In a further aspect, the present disclosure provides methods of treatment of a cancer comprising administration of a therapeutically effective amount of (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor. In a still further aspect, the cancer is AML.

### F. KITS

In a further aspect, the present disclosure relates to kits comprising at least one disclosed NAMPT inhibitor, or a pharmaceutically acceptable salt, hydrate, solvate, or polymorph thereof, and at least one HDAC8 inhibitor and/or at least one SIRT6 inhibitor, and and one or more of: (a) at least one agent known to treat a disorder associated with uncontrolled cellular proliferation activity; or (b) instructions for treating AML.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

### G. REFERENCES

References are cited herein throughout using the format of reference number(s) enclosed by parentheses corresponding to one or more of the following numbered references. For example, citation of references numbers 1 and 2 immediately herein below would be indicated in the disclosure as (Refs. 1 and 2).
1. Roboz G J. Epigenetic targeting and personalized approaches for AML. Hematology American Society of Hematology Education Program 2014; 2014:44-51.
2. Siegel R L, Miller K D, Jemal A. Cancer statistics, 2018. CA: a cancer journal for clinicians 2018; 68:7-30.
3. Martin P R, Shea R J, Mulks M H. Identification of a plasmid-encoded gene from Haemophilus ducreyi which confers NAD independence. J Bacteriol 2001; 183:1168-74.
4. Hasmann M, Schemainda I. FK866, a Highly Specific Noncompetitive Inhibitor of Nicotinamide Phosphoribosyltransferase, Represents a Novel Mechanism for Induction of Tumor Cell Apoptosis. Cancer Res 2003; 63:7436-42.
5. Mitchell S R, Larkin K, Grieselhuber N R, Lai T H, Cannon M, Orwick S, et al. Selective targeting of NAMPT by KPT-9274 in acute myeloid leukemia. Blood advances 2019; 3:242-55.
6. Chowdhry S, Zanca C, Rajkumar U, Koga T, Diao Y, Raviram R, et al. NAD metabolic dependency in cancer is shaped by gene amplification and enhancer remodelling. Nature 2019; 569:570-5.
7. Shats I, Williams J G, Liu J, Makarov M V, Wu X, Lih F B, et al. Bacteria Boost Mammalian Host NAD Metabolism by Engaging the Deamidated Biosynthesis Pathway. Cell metabolism 2020; 31:564-79 e7.
8. Thakur B K, Dittrich T, Chandra P, Becker A, Kuehnau W, Klusmann J H, et al. Involvement of p53 in the cytotoxic activity of the NAMPT inhibitor FK866 in myeloid leukemic cells. International journal of cancer Journal international du cancer 2013; 132:766-74.
9. Holen K, Saltz L B, Hollywood E, Burk K, Hanauske A R. The pharmacokinetics, toxicities, and biologic effects of FK866, a nicotinamide adenine dinucleotide biosynthesis inhibitor. Investigational new drugs 2008; 26:45-51.
10. Zabka T S, Singh J, Dhawan P, Liederer B M, Oeh J, Kauss M A, et al. Retinal toxicity, in vivo and in vitro, associated with inhibition of nicotinamide phosphoribosyltransferase. Toxicological sciences: an official journal of the Society of Toxicology 2015; 144:163-72.
11. Galli U, Colombo G, Travelli C, Tron G C, Genazzani A A, Grolla A A. Recent Advances in NAMPT Inhibitors: A Novel Immunotherapic Strategy. Frontiers in pharmacology 2020; 11:656.
12. Wang H, Diao D J, Shi Z C, Zhu X D, Gao Y W, Gao S R, et al. SIRT6 Controls Hematopoietic Stem Cell Homeostasis through Epigenetic Regulation of Wnt Signaling. Cell stem cell 2016; 18:495-507.
13. Mao Z, Hine C, Tian X, Van Meter M, Au M, Vaidya A, et al. SIRT6 promotes DNA repair under stress by activating PARP1. Science 2011; 332:1443-6.
14. Toiber D, Erdel F, Bouazoune K, Silberman D M, Zhong L, Mulligan P, et al. SIRT6 recruits SNF2H to DNA break sites, preventing genomic instability through chromatin remodeling. Molecular cell 2013; 51:454-68.
15. Hou T, Cao Z, Zhang J, Tang M, Tian Y, Li Y, et al. SIRT6 coordinates with CHD4 to promote chromatin relaxation and DNA repair. Nucleic acids research 2020; 48:2982-3000.
16. Maria Gkotzamanidou M S, JesGs Martin Senchez, Mehmet Kemal Samur, Stephane Minvielle, Florence Magrangeas, Herve Avet-Loiseau, Athanasios-Meletios Dimopoulos, Kenneth C. Anderson and Nikhil C. Munshi. HDAC8 is recruited to DNA double strand breaks sites and affects the homologous recombination efficiency in multiple myeloma. 2015; Philadelphia, P A. American Association for Cancer Research. p 30305.
17. Santos-Barriopedro I, Li Y, Bahl S, Seto E. HDAC8 affects MGMT levels in glioblastoma cell lines via interaction with the proteasome receptor ADRM1. Genes & cancer 2019; 10:119-33.
18. Long J, Jia M Y, Fang W Y, Chen X J, Mu L L, Wang Z Y, et al. FLT3 inhibition upregulates HDAC8 via FOXO to inactivate p53 and promote maintenance of FLT3-ITD+ acute myeloid leukemia. Blood 2020; 135:1472-83.
19. Ying S, Chen Z, Medhurst A L, Neal J A, Bao Z, Mortusewicz O, et al. DNA-PKcs and PARP1 Bind to Unresected Stalled DNA Replication Forks Where They Recruit XRCC1 to Mediate Repair. Cancer research 2016; 76:1078-88.
20. Bindra R S, Goglia A G, Jasin M, Powell S N. Development of an assay to measure mutagenic non-homologous end-joining repair activity in mammalian cells. Nucleic acids research 2013; 41:e115.
21. Walker L A, Sovic M G, Chiang C L, Hu E, Denninger J K, Chen X, et al. CLEAR: coverage-based limiting-cell experiment analysis for RNA-seq. Journal of translational medicine 2020; 18:63.
22. Costello R T, Mallet F, Gaugler B, Sainty D, Arnoulet C, Gastaut J A, et al. Human acute myeloid leukemia CD34+/CD38− progenitor cells have decreased sensitivity to chemotherapy and Fas-induced apoptosis, reduced immunogenicity, and impaired dendritic cell transformation capacities. Cancer research 2000; 60:4403-11.
23. Sanjana N E, Shalem O, Zhang F. Improved vectors and genome-wide libraries for CRISPR screening. Nature methods 2014; 11:783-4.
24. Wang B B, Wang M, Zhang W B, Xiao T F, Chen C H, Wu A, et al. Integrative analysis of pooled CRISPR genetic screens using MAGeCKFlute. Nature protocols 2019; 14:756-80.
25. Balasubramanian S, Ramos J, Luo W, Sirisawad M, Verner E, Buggy J J. A novel histone deacetylase 8 (HDAC8)-specific inhibitor PCI-34051 induces apoptosis in T-cell lymphomas. Leukemia 2008; 22:1026-34.
26. Tseng Y C, Kulp S K, Lai I L, Hsu E C, He W A, Frankhouser D E, et al. Preclinical Investigation of the Novel Histone Deacetylase Inhibitor AR-42 in the Treatment of Cancer-Induced Cachexia. Journal of the National Cancer Institute 2015; 107:djv274.
27. Liva S G, Coss C C, Wang J, Blum W, Klisovic R, Bhatnagar B, et al. Phase I study of AR-42 and decitabine in acute myeloid leukemia. Leukemia & lymphoma 2020:1-9.
28. Guzman M L, Yang N, Sharma K K, Balys M, Corbett C A, Jordan C T, et al. Selective activity of the histone deacetylase inhibitor AR-42 against leukemia stem cells: a novel potential strategy in acute myelogenous leukemia. Molecular cancer therapeutics 2014; 13:1979-90.
29. Mims A, Walker A R, Huang X, Sun J, Wang H, Santhanam R, et al. Increased anti-leukemic activity of decitabine via AR-42-induced upregulation of miR-29b: a novel epigenetic-targeting approach in acute myeloid leukemia. Leukemia 2013; 27:871-8.
30. Sulkowski P L, Corso C D, Robinson N D, Scanlon S E, Purshouse K R, Bai H W, et al. 2-Hydroxyglutarate produced by neomorphic IDH mutations suppresses homologous recombination and induces PARP inhibitor sensitivity. Science translational medicine 2017; 9.
31. Liva S G, Tseng Y C, Dauki A M, Sovic M G, Vu T, Henderson S E, et al. Overcoming resistance to anabolic SARM therapy in experimental cancer cachexia with an HDAC inhibitor. EMBO molecular medicine 2020; 12:e9910.
32. Lee J H, Choy M L, Ngo L, Foster S S, Marks P A. Histone deacetylase inhibitor induces DNA damage, which normal but not transformed cells can repair. Proceedings of the National Academy of Sciences of the United States of America 2010; 107:14639-44.
33. Adimoolam S, Sirisawad M, Chen J, Thiemann P, Ford J M, Buggy J J. HDAC inhibitor PCI-24781 decreases RAD51 expression and inhibits homologous recombination. Proceedings of the National Academy of Sciences of the United States of America 2007; 104:19482-7.
34. Inoue S, Li W Y, Tseng A, Beerman I, Elia A J, Bendall S C, et al. Mutant IDH1 Downregulates ATM and Alters DNA Repair and Sensitivity to DNA Damage Independent of TET2. Cancer cell 2016; 30:337-48.
35. Sulkowski P L, Oeck S, Dow J, Economos N G, Mirfakhraie L, Liu Y, et al. Oncometabolites suppress DNA repair by disrupting local chromatin signalling. Nature 2020.
36. Hanekamp D, Cloos J, Schuurhuis G J. Leukemic stem cells: identification and clinical application. International journal of hematology 2017; 105:549-57.
37. De Grandis M, Mancini S J, Aurrand-Lions M. In quest for leukemia initiating cells in AML. Oncoscience 2018; 5:9-10.
38. Viale A, De Franco F, Orleth A, Cambiaghi V, Giuliani V, Bossi D, et al. Cell-cycle restriction limits DNA damage and maintains self-renewal of leukaemia stem cells. Nature 2009; 457:51-6.
39. Mohrin M, Bourke E, Alexander D, Warr M R, Barry-Holson K, Le Beau M M, et al. Hematopoietic stem cell quiescence promotes error-prone DNA repair and mutagenesis. Cell stem cell 2010; 7:174-85.
40. Dai Y, Chen S, Kmieciak M, Zhou L, Lin H, Pei X Y, et al. The novel Chk1 inhibitor MK-8776 sensitizes human leukemia cells to HDAC inhibitors by targeting the intra-S checkpoint and DNA replication and repair. Molecular cancer therapeutics 2013; 12:878-89.
41. Bellio C, DiGloria C, Foster R, James K, Konstantinopoulos P A, Growdon W B, et al. PARP Inhibition Induces Enrichment of DNA Repair-Proficient CD133 and CD117 Positive Ovarian Cancer Stem Cells. Molecular cancer research: MCR 2019; 17:431-45.
42. Cagnetta A, Soncini D, Orecchioni S, Talarico G, Minetto P, Guolo F, et al. Depletion of SIRT6 enzymatic activity increases acute myeloid leukemia cells' vulnerability to DNA-damaging agents. Haematologica 2018; 103:80-90.
43. Onn L, Portillo M, Ilic S, Cleitman G, Stein D, Kaluski S, et al. SIRT6 is a DNA double-strand break sensor. eLife 2020; 9.
44. Van Meter M, Mao Z, Gorbunova V, Seluanov A. Repairing split ends: SIRT6, mono-ADP ribosylation and DNA repair. Aging 2011; 3:829-35.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### H. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Exemplary Studies

Nicotinamide phosphoribosyltransferase (NAMPT) inhibitors are currently in development, but may be limited as a single agent therapy due to compound-specific toxicity and cancer metabolic plasticity allowing resistance development. To potentially lower the dose of NAMPT inhibition required for therapeutic benefit against acute myeloid leukemia (AML) cells, we performed a genome-wide CRISPRi screen to identify rational disease-specific partners for a novel NAMPT inhibitor, KPT-2974.

In the present examples, cell lines and primary cells are analyzed for cell viability, self-renewal and responses at RNA and protein levels with loss-of-function approaches and pharmacologic treatments. In vivo efficacy of combination therapy is evaluated with xenograft model.

The data disclosed the present examples identify two histone deacetylases, HDAC8 and SIRT6, whose knockout conferred synthetic lethality with KPT-9274 in AML cells. Furthermore, HDAC8-specific inhibitor, PCI-34051, or clinical Class I HDAC inhibitor, AR-42, in combination with KPT-9274, synergistically decreased the survival of AML cells in a dose-dependent manner. AR-42/KPT-9274 co-treatment attenuated colony-forming potentials of AML patient cells while sparing healthy hematopoietic cells. Importantly, combined therapy demonstrated promising in vivo efficacy compared with KPT-9274 or AR-42 monotherapy. Mechanistically, genetic inhibition of SIRT6 potentiated KPT-9274 effect on PARP-1 suppression by abolishing mono-ADP ribosylation. AR-42/KPT-9274 co-treatment resulted in synergistic attenuation of homologous recombination (HR) and nonhomologous end joining (NHEJ) pathways in cell lines and leukemia initiating cells (LICs).

The present examples provide evidence that HDAC8 inhibitor or shSIRT6-induced deficiencies in DNA repair pathways is potently synergistic with NAMPT targeting, with minimal toxicity towards normal cells, providing a rationale for a novel combination-based treatment for AML.

### 2. Materials and Methods

Genomewide loss-of-function screen. The human GeCKO CRISPR knockout library was obtained from Addgene. The library was amplified in bacteria and packaged into viral particles in HEK293FT cells. MOLM13 cells were transduced with lentiviral particles at pre-determined ratio in the presence of polybrene and spinoculated at 450×g for 90 minutes. Puromycin selection was initiated after 48 hours and continued for 7 days to eliminate cells with essential gene-targeting single guide RNA (sgRNA) and non-transduced cells. The transduced cells were cultured for 3 days in the presence of 50 nM KPT-9274 or DMSO to negatively select sgRNAs that sensitize resistant cells to KPT-9274. Cells were collected on days 0 and 3 and subject to P5/P7 barcoding and NGS sequencing using Illumina HiSeq4000 sequencer to detect the abundance of each sgRNA. Sequencing data were analyzed using MAGeCK VISPR algorithm to discover sgRNAs that were negatively or positively selected in the presence of KPT-9274.

Drug materials. KPT-2974 was obtained from Selleckchem for in vitro study and Karyopharm Therapeutics for in vivo study. AR-42 and PCI-34051 were purchased from Selleckchem. For in vivo study, AR-42 was formulated in 0.5% methylcellulose [w/v] and 0.1% Tween-80 [v/v] in sterile water. Placebo for the in vivo KPT-2974 study was obtained from Karyopharm Therapeutics.

Cell lines and shRNA transfection. MOLM13 (DSMZ Cat #ACC-554, RRID:CVCL_2119), MV4-11 (RRID:CVCL_0064) and OCI-AML3 (DSMZ Cat #ACC-582, RRID:CVCL_1844) were purchased from DSMZ (Braunschweig, Germany) and cultured in RPMI1640 (Gibco) supplemented with 10% fetal bovine serum (FBS). Kasumi-1 cells (ATCC Cat #PTA-5077, RRID:CVCL_6911) were purchased from ATCC (Manassas, VA) and cultured in RPMI1640 (Gibco) supplemented with 20% FBS. Isocitrate dehydrogenase2 (IDH2)^{R141OQ} (ATCC Cat #CRL-20031G, RRID:CVCL_UE10) and wildtype (WT) TF-1 cells (ATCC Cat #CRL-2003, RRID:CVCL 0559) were obtained from ATCC (Manassas, VA) and cultured in RPM11640 (Gibco) supplemented with 10% FBS and 2 ng/ml recombinant human GM-CSF. MOLM13-luciferase cells were a kind gift from Dr. Ramiro Garzon (Ohio State University). HEK293FT cells were obtained from Life Technologies and cultured in DMEM (Gibco) with 10% FBS. All cell lines were used between passage three and twenty and routinely tested negative for mycoplasma contamination with Universal Mycoplasma Detection Kit (ATCC 30-1012K). Cells were authenticated with microsatellite genotyping (short tandem repeat analysis by the Ohio State University Genomic Services Core). AML patient and normal donor samples were obtained from The Ohio State University (OSU) Leukemia Tissue Bank under an institutional review board-approved protocol with informed consent according to the Declaration of Helsinki. For shRNA transfection, shRNA oligos for knockdown of SIRT6, HDAC8, DCPS, PTGS1, HEXA and IMPA2 in lentiviral vector pLKO.1 were purchased from Sigma (Sigma-Aldrich, St. Louis, MO). The following shRNA sequences were used:

shHDAC8-coding sequence (CDS):
(SEQ ID: 1)
CCGGGCGTATTCTCTACGTGGATTTCTCGAGAAATCCACGTAGAGAATACGCTTTTTG
(SEQ ID: 2)
CCGGGCGTATTCTCTACGTGGATTTCTCGAGAAATCCACGTAGAGAATACGCTTTTT
(SEQ ID: 3)
CCGGAGTCGCTGGTCCCGGTTTATACTCGAGTATAAACCGGGACCAGCGACTTTTTTG
(SEQ ID: 4)
CCGGTTACGATTGCGACGGAAATTTCTCGAGAAATTTCCGTCGCAATCGTAATTTTTG
shHDAC8-3′ untranslated region (3′ UTR):
(SEQ ID: 5)
CCGGTGACAGAAAGAGATCAGGTTTCTCGAGAAACCTGATCTCTTTCTGTCATTTTT
shSIRT6-3′ UTR:
(SEQ ID: 6)
CCGGCTCCCTGGTCTCCAGCTTAAACTCGAGTTTAAGCTGGAGACCAGGGAGTTTTTG
shDCPS-CDS:
(SEQ ID: 7)
CCGGGCAGTTCTCCAATGATATCTACTCGAGTAGATATCATTGGAGAACTGCTTTTTG
(SEQ ID: 8)
CCGGGGGAGACCATCTGCGAGTATACTCGAGTATACTCGCAGATGGTCTCCCTTTTTG
(SEQ ID: 9)
CCGGGCAGTTCTCCAATGATATCTACTCGAGTAGATATCATTGGAGAACTGCTTTTT
shDCPS-3′UTR:
(SEQ ID: 10)
CCGGCAGGCAGAAGAGCACAGATGTCTCGAGACATCTGTGCTCTTCTGCCTGTTTTTG
(SEQ ID: 11)
CCGGCAGGCAGAAGAGCACAGATGTCTCGAGACATCTGTGCTCTTCTGCCTGTTTTT
shPTGS1:
(SEQ ID: 12)
CCGGCGGCCACATTTATGGAGACAACTCGAGTTGTCTCCATAAATGTGGCCGTTTTTG
(SEQ ID: 13)
CCGGCGCAAGAGGTTTGGCATGAAACTCGAGTTTCATGCCAAACCTCTTGCGTTTTTG
(SEQ ID: 14)
CCGGACAATCTGGAGCGTCAGTATCCTCGAGGATACTGACGCTCCAGATTGTTTTTTG
(SEQ ID: 15)
CCGGCGTGAGCTATTACACTCGTATCTCGAGATACGAGTGTAATAGCTCACGTTTTTG
(SEQ ID: 16
GTACCGGGATGGTCTTAAATGCTCATTTCTCGAGAAATGAGCATTTAAGACCATCTTTTTTG
shHEXA:
(SEQ ID: 17)
CCGGCCCAGTCTCAATAATACCTATCTCGAGATAGGTATTATTGAGACTGGGTTTTT
(SEQ ID: 18)
CCGGCGTCCTTTACCCGAACAACTTCTCGAGAAGTTGTTCGGGTAAAGGACGTTTTT
(SEQ ID: 19)
CCGGGCGAGAGGATATTCCAGTGAACTCGAGTTCACTGGAATATCCTCTCGCTTTTT
(SEQ ID: 20)
CCGGGTTGGATACATCTCGCCATTACTCGAGTAATGGCGAGATGTATCCAACTTTTT
CCGGTATCTCCAAGGCGTTGGTATACTCGAGTATACCAACGCCTTGGAGATATTTTTG
shIMPA2:
(SEQ ID: 21)
CCGGGCTGTTCGACAAGAGCTTGAACTCGAGTTCAAGCTCTTGTCGAACAGCTTTTTG
(SEQ ID: 22)
CCGGGCCTTACAGACGATTAACTATCTCGAGATAGTTAATCGTCTGTAAGGCTTTTTG
(SEQ ID: 23)
CCGGGCTGCAGATCTTGTGACAGAACTCGAGTTCTGTCACAAGATCTGCAGCTTTTTG
(SEQ ID: 24)
GTACCGGACGTCTCTCTCACCAGGATTTCTCGAGAAATCCTGGTGAGAGAGACGTTTTTTTG
(SEQ ID: 25)
GTACCGGAGAGGGAGTTGTCACGCTACACTCGAGTGTAGCGTGACAACTCCCTCTTTTTTTG

All viruses were produced using the HEK293FT cells with packaging and envelope plasmids, psPax2 and VSVG. Cells were transduced by spin infection at 1,500 rpm for 90 minutes in the presence of polybrene. Seventy-two hours after transduction, puromycin was added to the culture to select shRNA-stable clones.

MTS assay. For MTS assays, AML cell lines or patient-derived cells were treated in a 96-well plate at 1-6×10⁵ cells per well for 24-72 hours. Patient-derived CD34⁺ primary cells were cultured in 96-well plates coated with collagen in the presence of 10 ng/ml IL-3, 10 ng/ml IL-6, 10 ng/ml SCF and 10 ng/ml GM-CSF. Cells were treated with increasing concentrations of KPT-9274 (0 nM to 10 μM) or vehicle control (dimethyl sulfoxide [DMSO]). In drug combination studies, a range of concentrations of AR-42 or PCI-34051 were added in combination with increasing concentrations of KPT-9274 (0 nM-10 μM). MTS [3-(4,5-dimethylthiazol-2-yl)-5-(3-carboxymethoxyphenyl)-2-(4-sulfophenyl)-2H-tetrazolium](Pro-mega, Madison, WI) was incubated with drug-treated cells according to the manufacturer's instructions. Plates were read by using a BioTek synergy H4 hybrid multimode microplate reader (Thermo Fisher Scientific) at 490 nM. Combenefit software was used to calculate synergy scores for pairwise combinations of drug doses based on the differences between the observed and expected levels of inhibition (assuming independent activities).

Colony-forming unit (CFU) assays. Age-matched cryopreserved primary cells from bone marrow and apheresis of AML patients with various co-mutations and CD34⁺ healthy bone marrow cells were acquired from Ohio State University Comprehensive Cancer Center Leukemia Tissue Bank. Viable cells were plated at optimal densities in duplicates in MethoCult H0435 without EPO (StemCell Technologies, Vancouver, Canada) in the presence of DMSO vehicle control, 0.1 μM KPT-9274, 0.8 μM AR-42, or combination of inhibitors. Formed colonies were counted blindly after 7-14 days and re-plated at 10,000 cells/well. The images of colonies were captured with Echo revolving microscope. The images of Giemsa staining of cytospin preparations were acquired by CX43 Olympus inverted microscope.

Flow cytometry for cell apoptosis, mitochondria membrane potential and DNA damage detection. Parental or shRNA-stable AML cell lines were plated at 5×10⁵ cells/mL and were treated with vehicle control (DMSO), 0.1 μM KPT-9274, 0.8 μM AR-42, or combined treatment for different time periods. Cells were washed and stained by using mouse anti-human Annexin V-fluorescein isothiocyanate and propidium iodide (PI) (BD Pharmingen). For mitochondrial depolarization probing, cells were stained with Tetramethylrhodamine, Methyl Ester, Perchlorate(TMRM)(Thermo Scientific) with Annexin V. For unrepaired DNA double-strand break detection, cells were fixed, permeabilized and stained with BV421 anti-H2A.X (pS139) (BD), APC anti-CD45 (BD) and near-IR live/dead dye (Thermo Scientific). Analysis was performed by using a Cytomics FC 500 Flow Cytometer (Beckman Coulter, Brea, CA), and data analysis was performed with Kaluza Analysis software (Gallios™ Kaluza, RRID:SCR_016700) (Beckman Coulter, Brea, CA, USA).

EJDR reporter assay. The EJDR assay was described previously (20). EJDR reporter cells were plated at 5×10⁵ cells per well in 10% FBS and RPM11640. After drug treatments, cells were grown in 10% Tet-free FBS (100-800; Gemini) and RPM11640. Incorporated I-Sce1 was induced with Shield1 (632189; Clontech) and triamcinolone (TA) (T6510; Sigma Aldrich) ligands for 24 hours. NHEJ and HR repair activities were assessed 48 hours post induction by quantification of DsRed- and GFP-positive cells on Cytomics FC 500 Flow Cytometer (Beckman Coulter, Brea, CA).

Limiting cell-RNA-seq (LC-RNA-seq) for primary patient LICs. LC-RNA-seq was performed as previously described to determine transcriptome profiles and differentially expressed genes (DEGs) of patient LICs treated with various inhibitors (21). Briefly, patient cells were cultured in the presence of cytokines and treated with vehicle, single agents or drug combination. 300 viable LICs from treated patient samples were sorted directly into SMART-seq lysis buffer with FACSAria Fusion (BD) based on putative stem cell markers CD34⁺ CD38⁻ (22). Initiating/stem cells were enriched in sorted CD34⁺ CD38⁻ cells, as CD34⁺ CD38⁻ subpopulations grew significantly more colonies than CD34⁺ CD38⁺ subpopulations in CFU assays (Unpublished observation). The Clontech SMARTer v4 kit (Takara Bio USA, Inc., Mountain View, CA) was used for preamplifying samples prior to library construction with the Nextera XT DNA Library Prep Kit (Illumina, Inc., San Diego, CA). Samples were sequenced to a depth of 15-20 million 2×150 bp clusters with the Illumina HiSeq 4000 platform. After quality control with CLEAR selection process and size-normalization, DEGs were called with DESeqs with (FDR) adjusted p-value <0.05. Principle component analysis (PCA) plots were created from count tables which were normalized by size and transformed. For pathway analysis, the list of DEGs, containing gene IDs and corresponding expression values, was uploaded into the IPA software (Qiagen) (Ingenuity Pathway Analysis, RRID:SCR_008653). The “core analysis” function in the software was employed to interpret the differentially expressed data and identify top enriched pathways, which contained canonical pathways, gene network and upstream regulators.

In vivo MOLM13 xenograft study. All experiments were approved by the OSU Institutional Animal Care and Use Committee. Male NOD-Prkdc^{em26cd52}Il2rg^{em26cd22}/NjuCrl (NCG) mice (RRID:IMSR_CRL:572) (aged 6-8 weeks) were obtained from Charles River Laboratory. Mice were group-housed under conditions of constant photoperiod (12-hour light/12-hour dark), temperature, and humidity with adlibitum access to water and irradiated standard pelleted chow. 104 luciferase-tagged MOLM13 cells were injected via tail vein into NCG mice. On day 5 post-engraftment, mice were randomized to receive placebo (vehicle; Karyopharm Therapeutics) once daily, 100 mg/kg of KPT-9274 (Karyopharm Therapeutics) once daily, 20 mg/kg AR-42 every another day and the combination of KPT-9274 and AR-42 via oral gavage. Overall survival was the primary end point for the majority of the mice. A separate cohort of mice per group was used to monitor leukemic progression using IVIS imaging. Mice were monitored by animal technicians who were blinded to the treatment groups and determined when mice met early removal criteria (20% weight loss, lethargy, hunching, and poor body condition). Mice were euthanized by CO₂ inhalation.

Statistical analysis. Data are presented as the mean±SEM of independent experiments, unless otherwise specified. Statistical analyses were performed with GraphPad Prism 7.0 (GraphPad Prism, RRID:SCR_002798) or SAS/STAT software (version 9.0) with ANOVA with Tukey's post-test correction for multigroup comparisons or a 2-tailed Student's t test for 2-group comparisons, unless otherwise specified in the FIG. legends. A p-value of less than 0.01 or 0.05 was considered significant. With 8 mice/group there is 80% power to detect a difference between treated vs control group at a 5% significance level. For survival analyses, a Cox proportional hazard model was used to determine statistical significance. For CFU measurements, a negative binomial model was used to fit data.

### 3. Results

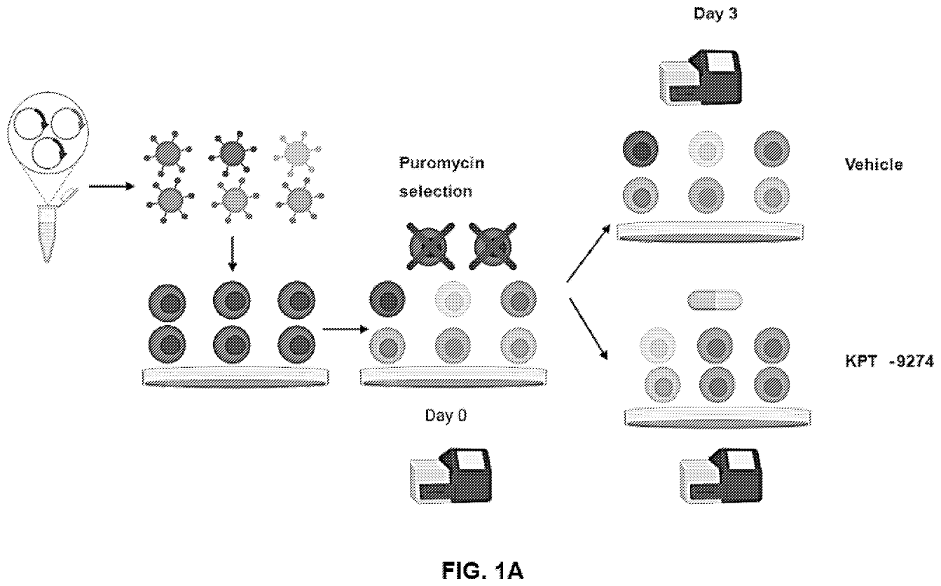

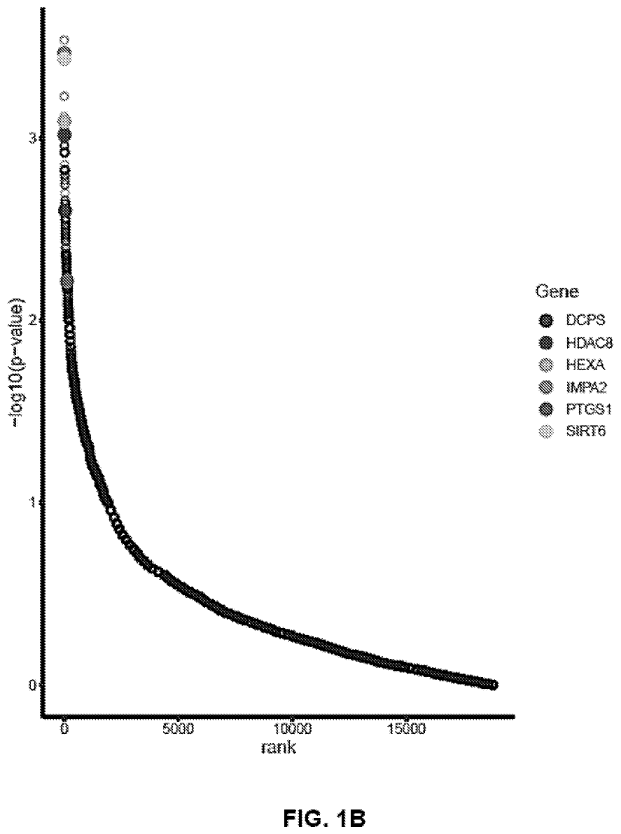

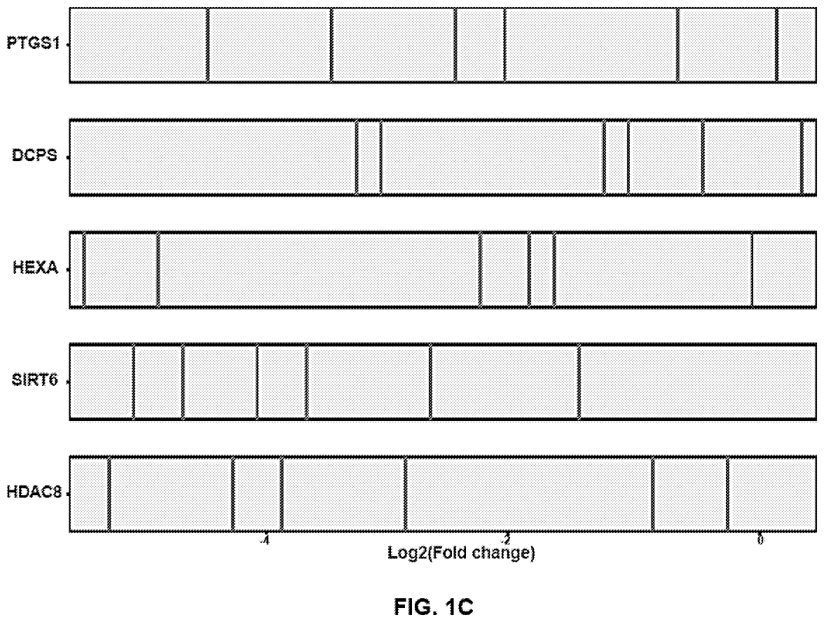

Histone deacetylases, HDAC8 and SIRT6, are targets for synthetic lethality with NAMPT inhibitor, KPT-9274, in genome-wide CRISPR screens in AML cells. To perform the genome-wide CRISPR-Cas9 screen, this study used the human lentivirus-based GeCKO library, which contains ˜130,000 sgRNAs targeting ˜20,000 protein-coding genes and microRNAs (Ref. 23; ˜6 sgRNAs per gene). The library was packaged and transduced into Cas9-expressing MOLM13 cells (Ref. 5). Then, transduced cells were selected in puromycin and received sublethal dose of 50 nM KPT-9274 or DMSO control for 3 days. Genomic DNA was isolated from cells, and deep-sequenced to measure read counts of each sgRNA. Changes in abundance of each sgRNA were assessed using the MAGeCK program (Ref. 24; FIG. 1A). 400× coverage of the library was achieved with 95% of the sgRNA sequences were retained in all samples, ensuring the sufficient library coverage. By analyzing the sgRNAs that were negatively selected in the presence of KPT-9274 with MAGeCK algorithm, several genes were identified as high-quality hits with small p-values (FIG. 1B and Table 1). The data allowed prioritizing top hits with available preclinical or clinical inhibitors (i.e. HDAC8, SIRT6, IMPA2, PTGS1 and DCPS) for validation. High proportion of sgRNAs targeting these genes experienced large fold depletion following KPT-9274 treatment (FIG. 1C). In this unbiased manner, gene candidates that could serve to enhance the therapeutic index and overcome NAMPT inhibitor insensitivity were identified. The RRA model used herein integrates the sgRNA-level fold change and p-values to identify potentially interesting gene hits. The RRA enrichment score and p-value for each gene are shown below in Table 1.

TABLE 1
List of CRISPR screen top ranked hits
calculated with MAGeCK VISPR.*

| Rank | Genes | score | p-value |
|---|---|---|---|
| 1 | CEP41 | 2.70E−05 | 0.00016 |
| 2 | C11orf52 | 3.40E−05 | 0.00019 |
| 3 | PRKAR2B | 4.60E−05 | 0.00023 |
| 4 | ARHGEF10 | 4.70E−05 | 0.00023 |
| 5 | TOMM5 | 5.90E−05 | 0.00029 |
| 6 | IMPA2 | 7.00E−05 | 0.00034 |
| 7 | SIRT6 | 7.70E−05 | 0.00037 |
| 8 | XKRY | 0.0001 | 0.00051 |
| 9 | ZNF343 | 0.00012 | 0.00058 |
| 10 | KLHL2 | 0.00012 | 0.00059 |
| 11 | KCNH2 | 0.00013 | 0.0006 |
| 12 | PRSS42 | 0.00015 | 0.00076 |
| 13 | MSN | 0.00015 | 0.00076 |
| 14 | TM6SF2 | 0.00016 | 0.0008 |
| 15 | HEXA | 0.00016 | 0.00081 |
| 16 | OR10H4 | 0.00017 | 0.00087 |
| 17 | HDAC8 | 0.00018 | 0.00096 |
| 18 | DUSP12 | 0.0002 | 0.001 |
| 19 | SEMA4C | 0.00024 | 0.0011 |
| 20 | LSMEM1 | 0.00025 | 0.0012 |
| 21 | PDIA6 | 0.00028 | 0.0012 |
| 22 | CRIP1 | 0.00029 | 0.0012 |
| 23 | HPSE | 0.0003 | 0.0012 |
| 24 | ZNF454 | 0.00032 | 0.0014 |
| 25 | CNTN6 | 0.00034 | 0.0015 |
| 26 | KMT2B | 0.00034 | 0.0015 |
| 27 | DNAJB7 | 0.00036 | 0.0016 |
| 28 | SLC17A2 | 0.00036 | 0.0016 |
| 29 | ACYP2 | 0.0004 | 0.0018 |
| 30 | OR4C16 | 0.00048 | 0.0022 |
| 31 | SLC41A2 | 0.00048 | 0.0022 |
| 32 | DCPS | 0.00053 | 0.0025 |
| 33 | CSF3R | 0.00057 | 0.0025 |
| 34 | CTSG | 0.00058 | 0.0028 |
| 35 | MOB1B | 0.00059 | 0.0028 |
| 36 | ZNF837 | 0.00065 | 0.0032 |
| 37 | FAM214A | 0.00067 | 0.0033 |
| 38 | NDUFA11 | 0.00068 | 0.0033 |
| 39 | UBE2J2 | 0.00071 | 0.0035 |
| 40 | RBAK | 0.00072 | 0.0036 |
| 41 | PDZK1IP1 | 0.00074 | 0.0037 |
| 42 | SLC25A6 | 0.00077 | 0.004 |
| 43 | MYO5A | 0.00079 | 0.0043 |
| 44 | BSN | 0.00085 | 0.0044 |
| 45 | ASF1A | 0.00085 | 0.0044 |
| 46 | MUC12 | 0.00087 | 0.0044 |
| 47 | KIAA1161 | 0.00092 | 0.0045 |
| 48 | CLPB | 0.0011 | 0.0051 |
| 49 | CXorf40B | 0.0011 | 0.0052 |
| 50 | BCL9 | 0.0011 | 0.0054 |
| 51 | GJC1 | 0.0011 | 0.0054 |
| 52 | CACNG3 | 0.0012 | 0.0057 |
| 53 | ATL3 | 0.0012 | 0.0061 |
| 54 | MTL5 | 0.0012 | 0.0061 |
| 55 | PTGS1 | 0.0012 | 0.0061 |
| 56 | ADAT2 | 0.0013 | 0.0061 |
| 57 | RASGRP2 | 0.0013 | 0.0061 |
| 58 | MRPL38 | 0.0013 | 0.0063 |
| 59 | ZNF224 | 0.0014 | 0.0067 |
| 60 | ZNF564 | 0.0015 | 0.0068 |
| 61 | HCN1 | 0.0015 | 0.0068 |
| 62 | PSEN2 | 0.0015 | 0.0071 |
| 63 | OR4D10 | 0.0015 | 0.0071 |
| 64 | ACVRL1 | 0.0015 | 0.0078 |
| 65 | STX5 | 0.0015 | 0.0081 |
| 66 | VSIG4 | 0.0015 | 0.0081 |
| 67 | ZNF778 | 0.0015 | 0.0081 |
| 68 | SLC35E3 | 0.0016 | 0.0081 |
| 69 | SLC30A9 | 0.0017 | 0.0086 |
| 70 | IL9 | 0.0017 | 0.0086 |
| 71 | IL1A | 0.0017 | 0.0087 |
| 72 | FAM19A2 | 0.0017 | 0.0087 |
| 73 | HOXD11 | 0.0018 | 0.0089 |
| 74 | KLHL18 | 0.0018 | 0.009 |
| 75 | CAP1 | 0.0018 | 0.0091 |
| 76 | GABRA3 | 0.0018 | 0.0091 |
| 77 | MTMR4 | 0.0018 | 0.0091 |
| 78 | C19orf40 | 0.0018 | 0.0092 |
| 79 | GPRC5B | 0.0018 | 0.0092 |
| 80 | CNTN2 | 0.0019 | 0.0096 |
| 81 | CHODL | 0.0021 | 0.0091 |

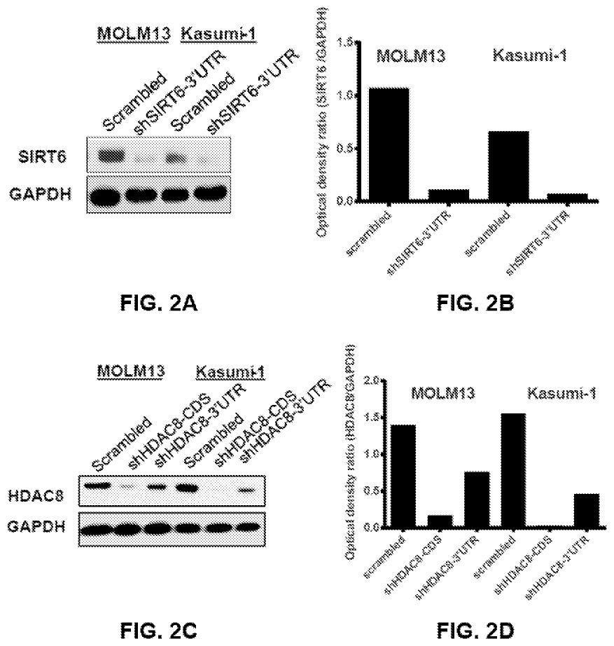

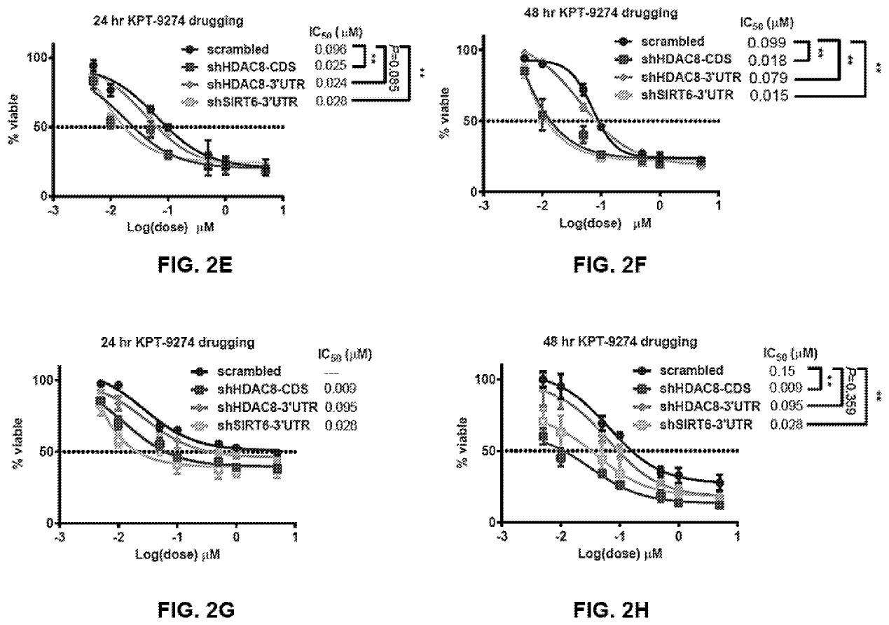

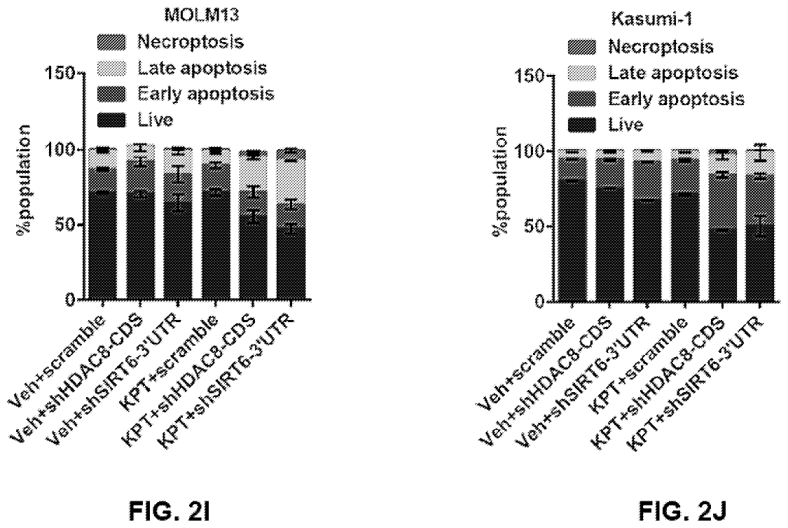

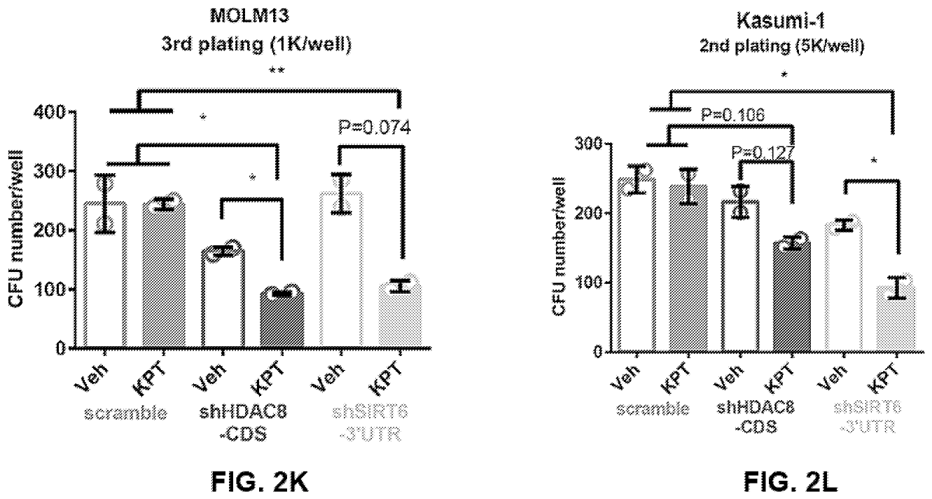

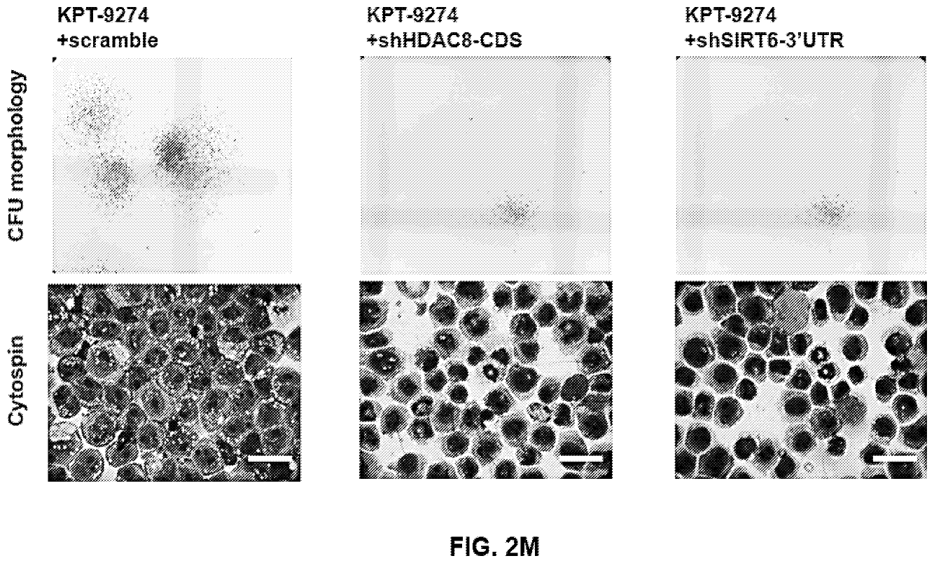

Genetic depletion of histone deacetylases sensitizes AML cells to NAMPT inhibition. To validate the results of the CRISPR screen, it was it was decided to first demonstrate genetically the cell-essential nature of the dropout genes on KPT-9274-sensitive cell line, MOLM13, and KPT-9274-insensitive cell line, Kasumi-1. A gene-by-gene knockout approach using the pLKO-shRNA system was employed. Four CDS-targeting shRNAs and one 3′UTR-targeting shRNA were designed per gene. CDS-targeting oligos were pooled and packaged into lentivirus. After 3 days of puromycin selection, HDAC8-CDS targeting shRNAs effectively reduced protein expression in Kasumi-1 and MOLM13 cells, while 3′UTR-targeting oligo only marginally decreased HDAC8 abundance (FIGS. 2A-2D). SIRT6 shRNAs showed the opposite properties: 3′UTR-targeting oligos decreased protein level by 80%. The knockdown efficiency can be maintained for at least 1 month in vitro.

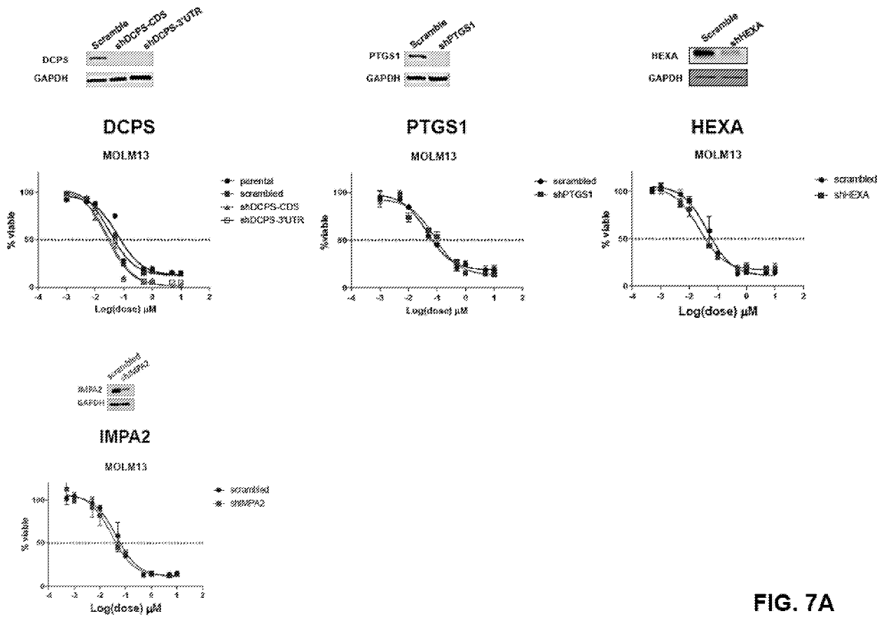

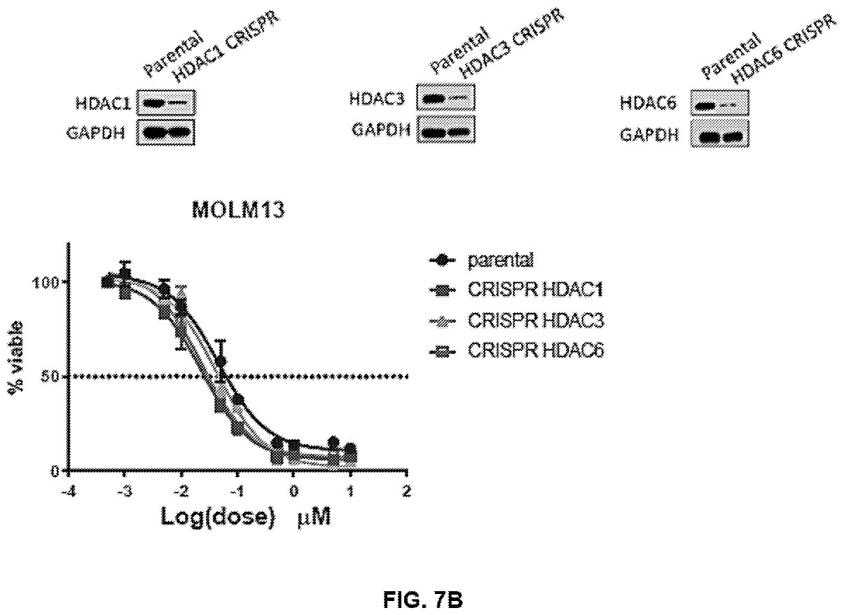

To determine whether the effects induced by downregulation of those targets play synergistic roles with NAMPT inhibition, we treated shHDAC8 and shSIRT6-stable cell lines with KPT-9274 at doses ranging from 0.005-5 μM for 24 and 48 hour and measured cell viability with MTS assays. shHDAC8-CDS and shSIRT6-3′UTR transfection reduced IC₅₀ doses of KPT-9274 for MOLM13 (at 48 hour, shHDAC8-CDS vs scramble: −0.069<95% CI<−0.046, p<0.001; shSIRT6-3′UTR vs scramble: −0.069<95% CI<−0.048, p<0.001), and Kasumi-1 (at 48 hour, shHDAC8-CDS vs scramble: −0.084<95% CI<−0.049, p<0.001; shSIRT6-3′UTR vs scramble: −0.083<95% CI<−0.03, p<0.001), resulting in a significant reduction of cell survival (FIGS. 2E-2H). The vulnerabilities of shHDAC8-3′UTR-transfected cells to KPT-9274 treatment were comparable with those of scramble-transfected cells, mirroring the low knockdown efficiency of this shRNA construct. These results imply that genetic depletion of HDAC8 decreased tumor survival in the presence of KPT-9274 proportional to the reduction in HDAC8. Knockdown of IMPA2, HEXA, PTGS1 and DCPS did not demonstrate strong synergies with KPT-9274 treatments, suggesting that they might be false positive hits from the screen (FIG. 7A).

To determine whether KPT-9274 and shRNA-mediated genetic depletion had synergistic effects on cell apoptosis, HDAC8 and SIRT6-depleted MOLM13 and Kasumi-1 cells were treated with KPT-9274 at sublethal IC₂₀ doses. In vehicle-treated cells, shRNA knockdown of HDAC8 and SIRT6 had a modest effect on cell apoptosis (FIGS. 2I-2J), whereas HDAC8 depletion in the presence of KPT-9274 resulted in a significantly increase in the percentage of apoptotic cells in MOLM13 and Kasumi-1 cell lines. Likewise, SIRT6 depletion and KPT-9274 cooperate to enhance apoptosis of AML cells. These results provided additional evidence of histone deacetylase dependency of KPT-9274-treated AML cells for survival.

Next, the serial replating potentials of shRNA-stable Kasumi-1 and MOLM13 cells were examined following inhibition of NAMPT in CFU assays. In the scramble groups, the colony numbers formed by cells treated with sublethal doses of KPT-9274 or vehicle control were comparable. On the contrary, in HDAC8 or SIRT6-depleted MOLM13 and Kasumi-1 cells, KPT-2974 treatment suppressed colony formation and decreased long-term self-renewal cell populations (FIGS. 2K-2L) (MOLM13: shHDAC8-CDS vs scramble p<0.05; shSIRT6-3′UTR vs scramble p<0.05; Kasumi-1: shHDAC8-CDS vs scramble p=0.106; shSIRT6-3′UTR vs scramble p<0.05). Knockdown of HDAC8 or SIRT6 altered the morphology of colonies in the presence of KPT-9274 by decreasing the size of colonies and rendering them more compact (FIG. 2M). To evaluate the effect of dual inhibition on cell differentiation, cytospin preparations of cells derived from CFU assays were stained. KPT-9274 treatment and depletion of HDAC8 or SIRT6 cooperatively differentiated AML cells (FIG. 2M). In addition, the blasts morphologically became more mature with condensed chromatin and fragmented nuclei.

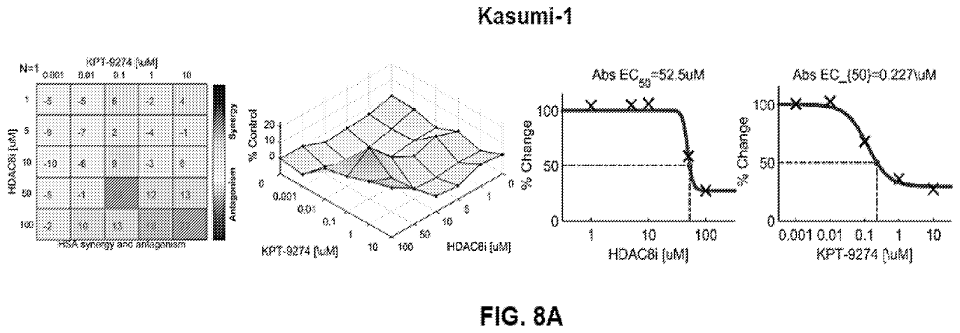

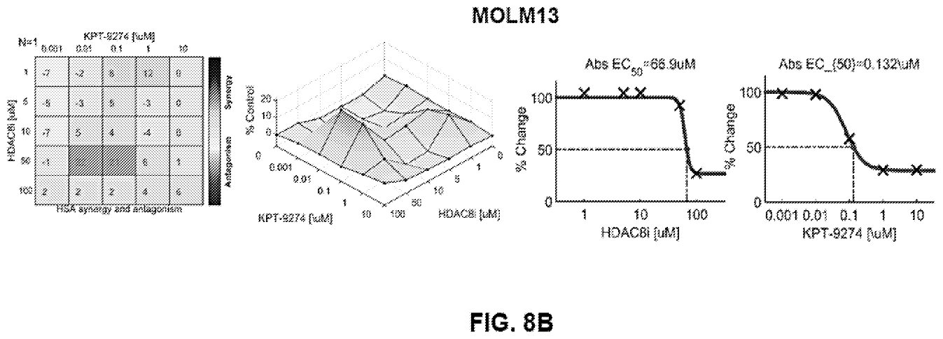

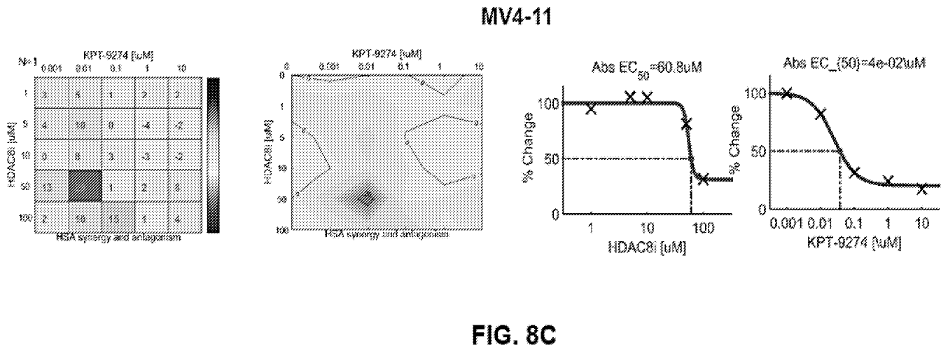

Pharmacological inhibition of HDAC8 confers vulnerability of AML subtypes to NAMPT inhibitor-induced cytotoxicity. To find translational relevance, HDAC8 with PCI-34051 (Ref. 25) were pharmacologically inhibited and potential synergistic effects of this inhibitor with KPT-9274 on AML cells were assessed. PCI-34051 exhibited >200-fold selectivity for HDAC8 over other HDAC isoforms (25). PCI-34051 induces caspase-dependent apoptosis in cell lines derived from T-cell lymphomas or leukemias (GI₅₀=2.4-4 μM), but not in other hematopoietic cells (Ref. 25). The data show that PCI-34051 and KPT-9274 interacted synergistically to reduce cell survival, as assessed by the HSA independence model (FIGS. 8A-8C). Drug synergistic effect was most pronounced for 50 μM PCI-34051 in combination with 0.01 μM KPT-9274 in MV4-11 cells and 0.1 μM KPT-9274 in Kasumi-1 cells. MOLM13 cells exhibited synergy over a broader range, as the combination of PCI-34051 at 50 μM and KPT-9274 at doses from 0.01 μM to 0.1 μM displayed the strongest synthetic lethality. It was reported that unlike the pan-HDAC inhibitor PCI-24781, PCI-34051 at a concentration as high as 50 μM, did not induce significant tubulin acetylation, implying that it did not exert its cytotoxicity through inhibition of other HDAC isoforms (Ref. 25). These data provide evidence that HDAC8 inhibition functions synergistically with NAMPT Inhibition to reduce AML viability. Nevertheless, the human equivalent dose of 50 μM of PCI-34051 is not considered to be a clinically tolerable dose. It was reported that PCI-34051 failed to induce AML death at low micromolar doses, although it caused apoptosis on T cell leukemia in a caspase-dependent manner (Ref.25). Without wishing to be bound by a particular theory, it is possible that PCI-34051 was not metabolized effectively in AML to hit HDAC8 targets.

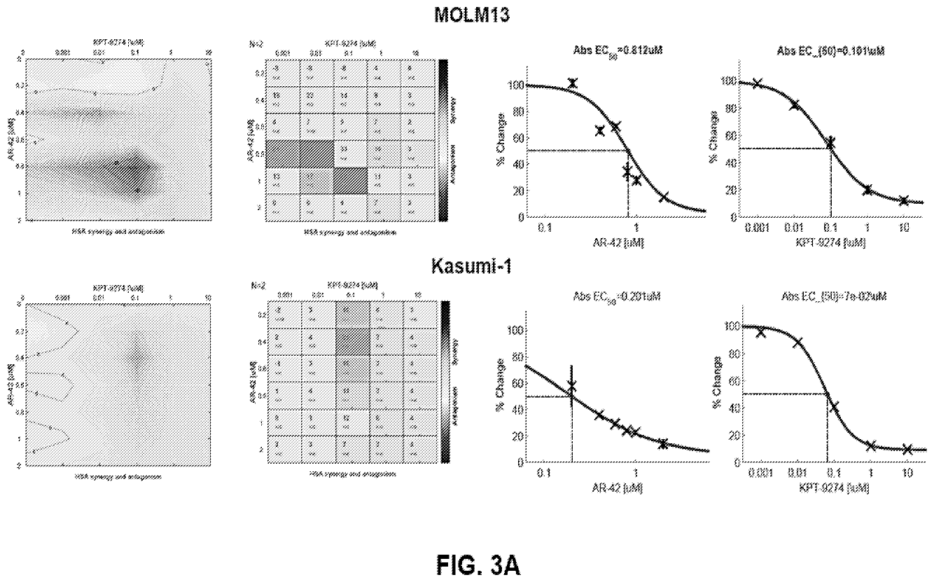

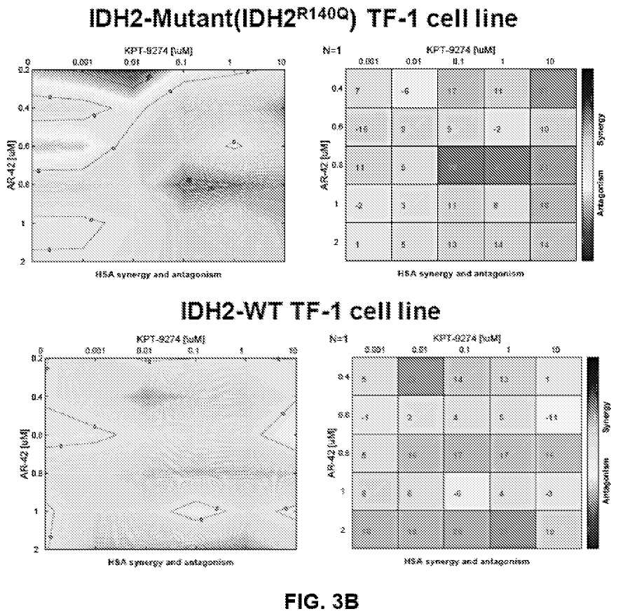

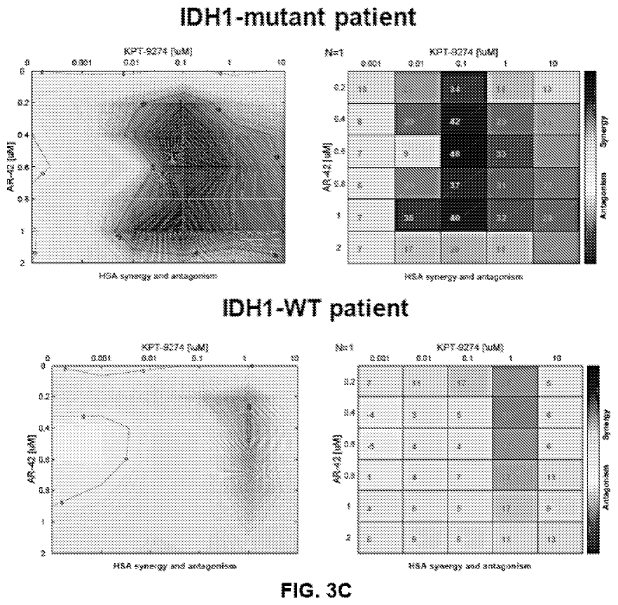

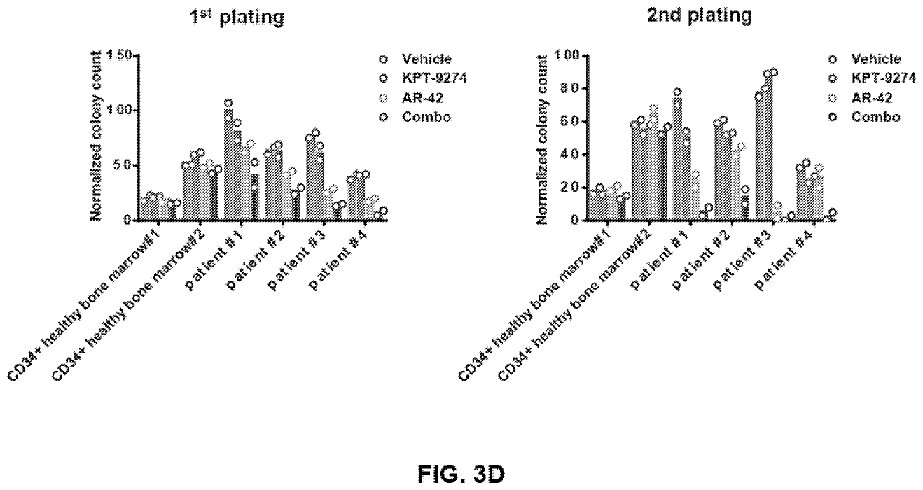

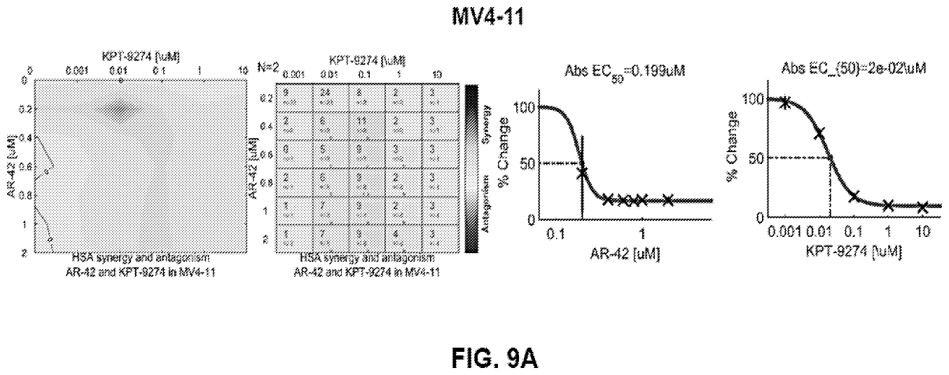

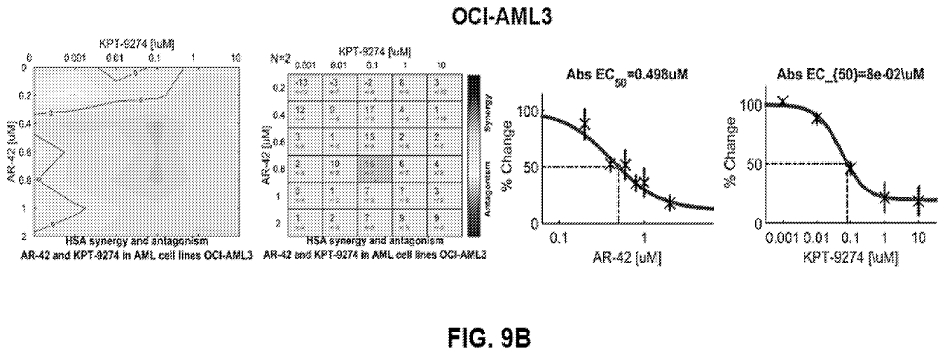

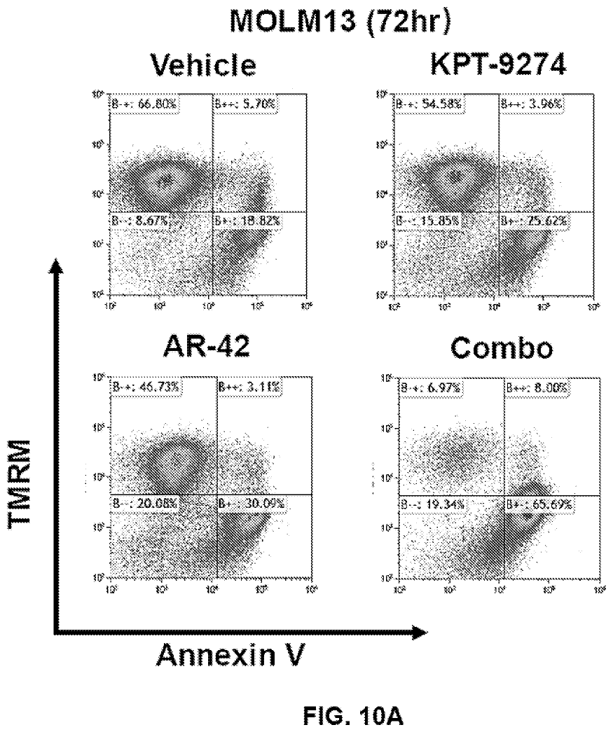

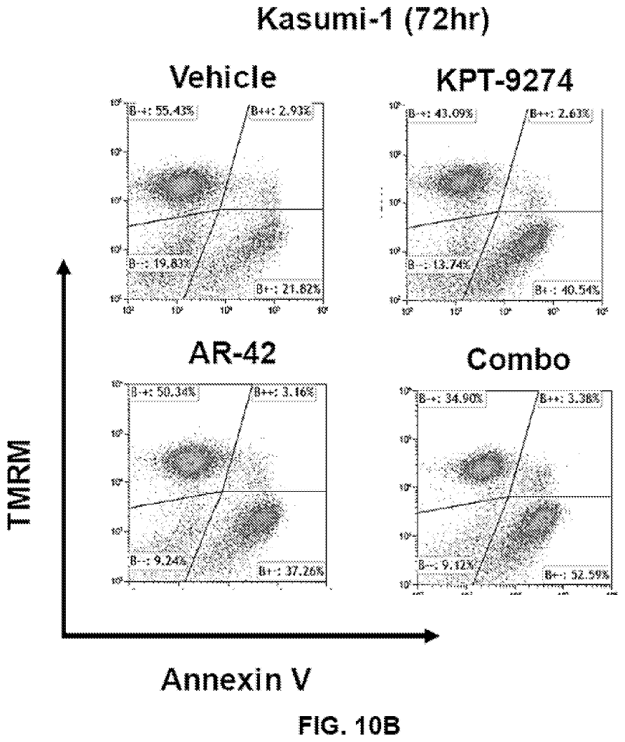

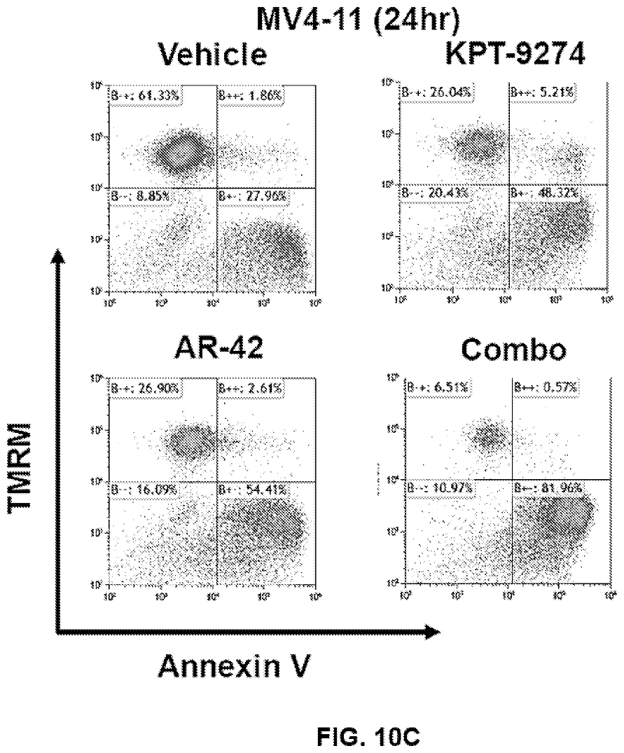

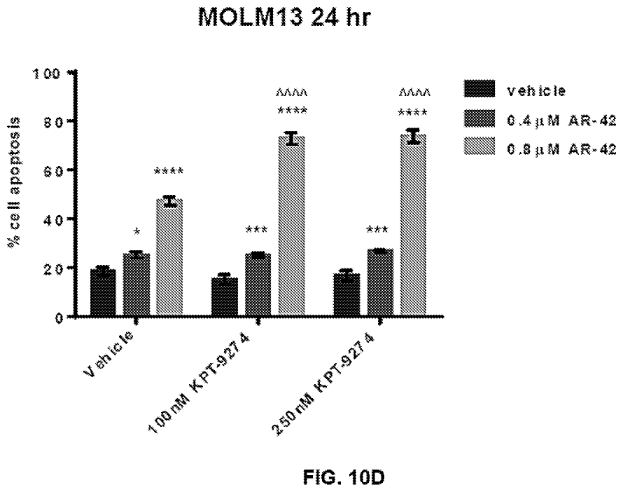

AR-42 is a potent pan-HDAC inhibitor of class I/II HDAC isoforms including HDAC8 (Ref. 26). AR-42 entered phase I clinical trials in combination with decitabine in AML and was shown to eradicate leukemia stem cells (Refs. 27-29). Depletion of HDAC1, HDAC3 or HDAC6 with CRISPR did not have significant impact on KPT-9274 sensitivity (FIG. 7B). This excluded the possible involvement of other HDAC isoforms in AR-42 efficacy. Therefore, AR-42 was employed as a tool compound to delineate the effect of HDAC8 inhibition on sensitivity of AML to KPT-9274 treatment. The highest synergistic scores were observed at combination doses of 0.1 μM KPT-9274 and 0.8 μM AR-42 for MOLM13 and of 0.1 μM KPT-9274 and 0.4 μM AR-42 for Kasumi-1 (FIG. 3A). Co-treatment of KPT-9274 and AR-42 also exhibited synergy on other cell lines, although to a lesser extent (FIGS. 9A-9B). To determine the effect of combined treatment on mitochondria respiration, the mitochondria membrane potential of treated cells was measured with TMRM staining. In comparison with single agents, combined treatment attenuated TMRM fluorescence intensity and concomitantly elevated Annexin V staining in MOLM13, Kasumi-1 and MV4-11 (MOLM13: combination vs AR-42 p<0.01; combination vs KPT-9274 p<0.01; combination vs vehicle p<0.01; Kasumi-1: combination vs AR-42 p<0.05; combination vs KPT-9274 p<0.05; combination vs vehicle p<0.05; MV4-11: combination vs AR-42 p<0.05; combination vs KPT-9274 p=0.0672; combination vs vehicle p<0.01) (Supplementary FIG. S4A-C). This suggests that disruption of mitochondria function may account for the observed synergism between KPT-9274 and AR-42 on AML. AR-42 and KPT-9274 co-treatment also increased the percentage of apoptotic cells in a dose-dependent manner (FIG. 10D).

It was reported that IDH1 and IDH2 mutations inhibits BRCA1/2 protein expression and induces HR repair defects by producing 2-hydroxyglutarate (2HG), leading to synthetic lethality triggered by PARP inhibitors (Ref. 30). The effect of IDH1 or IDH2 mutation on cellular sensitivity to combined treatment of KPT-9274 and AR-42 was evaluated by employing IDH2-mutant and -WT TF-1 cell lines as testing systems. The most synergistic area was achieved when 0.1-1 μM KPT-9274 was combined with 0.8 μM of AR-42 in IDH2-mutant cells. IDH2-mutant cells were more sensitive to the combination treatment than IDH2-WT cells, suggesting that co-treatment with AR-42 and KPT-9274 resulted in a synergistic cell death in a IDH2-dependent manner (FIG. 3B). We then tested the ability of the drug combination to inhibit the growth of primary AML patient cells. In accordance with findings in cell lines, exposure to AR-42 sensitized primary AML cells to KPT-9274 treatment (FIG. 3C). Combination treatment resulted in higher maximum synergy scores in IDH1-mutant patient cells than in IDH1-WT patient cells (max synergy score: 48 vs 26).

To further characterize the synergistic effect of combined treatment on the functional subsets of AML cells, methylcellulose colony-forming unit (CFU) assays were performed to determine whether drug combination would affect colony-forming capacities of primary cells. Compared with single agents, the drug combination significantly diminished the ability of primary AML blasts to form colonies and long-term replating potentials for cells from most patients (FIG. 3D; Table s2a and b). In contrast, the colony-forming capacities of CD34⁺ hematopoietic stem cells from the bone marrow of age-matched healthy donors were not substantially affected by combined treatment. Therefore, AR-42 and KPT-9274 synergistically reduced self-renewal capacities in AML cells while sparing normal human bone marrow cells.

TABLE 2a
Statistical analysis of CFU 1st plating of primary cells receiving different treatments
A negative binomial model was fit to the count data.
Estimates are comparisons are shown below.
Model estimates:

| | AML patients (n = 4) | | | Healthy donors (n = 2) | | |
|---|---|---|---|---|---|---|
| | Estimated | | | Estimated | | |
| Comparison | ratio | 95% CI | p-value | ratio | 95% CI | p-value |
| Combo vs. | 0.35 | (0.29, 0.43) | <.001 | 0.73 | (0.56, 0.96) | 0.026 |
| KPT-9274 | | | | | | |
| Combo vs. | 0.58 | (0.47, 0.71) | <.001 | 0.91 | (0.69, 1.21) | 0.509 |
| AR-42 | | | | | | |
| Combo vs. | 0.31 | (0.26, 0.38) | <.001 | 0.85 | (0.64, 1.12) | 0.238 |
| Vehicle | | | | | | |

Table 2a, continued (Direct comparison between AML patients and healthy donors).

| | Estimated | | |
|---|---|---|---|
| Comparison | ratio | 95% CI | p-value |
| Combo vs. KPT-9270 in AML vs. healthy | 0.48 | (0.35, 0.68) | <.001 |
| Combo vs. A-42 in AML vs. healthy | 0.63 | (0.45, 0.9) | 0.013 |
| Combo vs. Vehicle in AML vs. healthy | 0.37 | (0.26, 0.52) | <.001 |

TABLE 2b
Statistical analysis of CFU 2nd plating of primary cells receiving different treatments
A negative binomial model was fit to the count data.
Estimates are comparisons are shown below.
Model estimates:

| | AML patients (n = 4) | | | Healthy donors (n = 2) | | |
|---|---|---|---|---|---|---|
| | Estimated | | | Estimated | | |
| Comparison | ratio | 95% CI | p-value | ratio | 95% CI | p-value |
| Combo vs. | 0.11 | (0.08, 0.16) | <.001 | 0.94 | (0.63, 1.4) | 0.753 |
| KPT-9274 | | | | | | |
| Combo vs. | 0.21 | (0.14, 0.31) | <.001 | 0.8 | (0.54, 1.19) | 0.258 |
| AR-42 | | | | | | |
| Combo vs. | 0.1 | (0.07, 0.14) | <.001 | 0.87 | (0.58, 1.29) | 0.468 |
| Vehicle | | | | | | |

Table 2b, continued (Direct comparison between AML patients and healthy donors).

| | Estimated | | |
|---|---|---|---|
| Comparison | ratio | 95% CI | p-value |
| Combo vs. KPT-9270 in AML vs. healthy | 0.12 | (0.07, 0.2) | <.001 |
| Combo vs. A-42 in AML vs. healthy | 0.26 | (0.15, 0.45) | <.001 |
| Combo vs. Vehicle in AML vs. healthy | 0.11 | (0.06, 0.19) | <.001 |

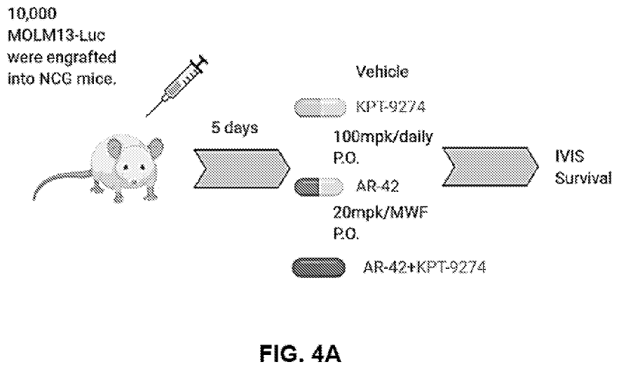

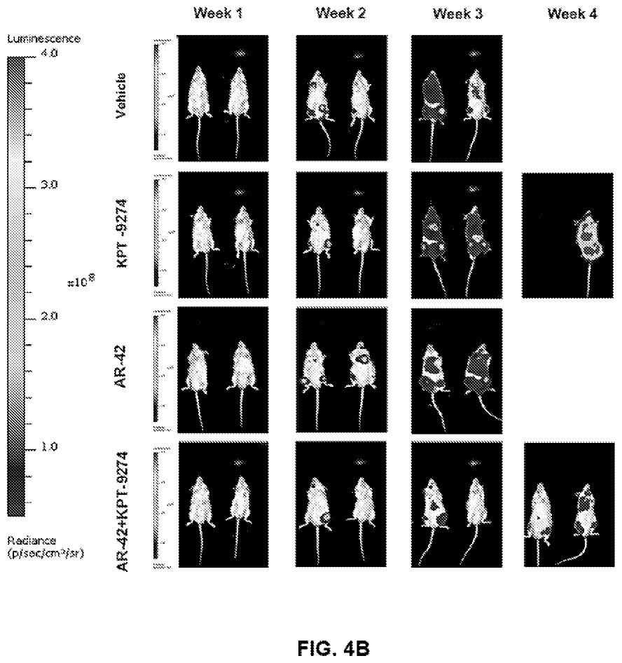

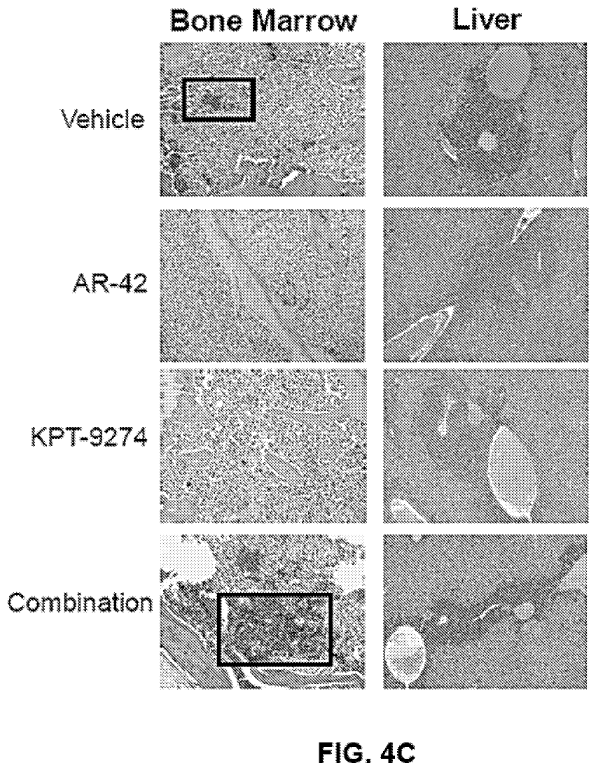

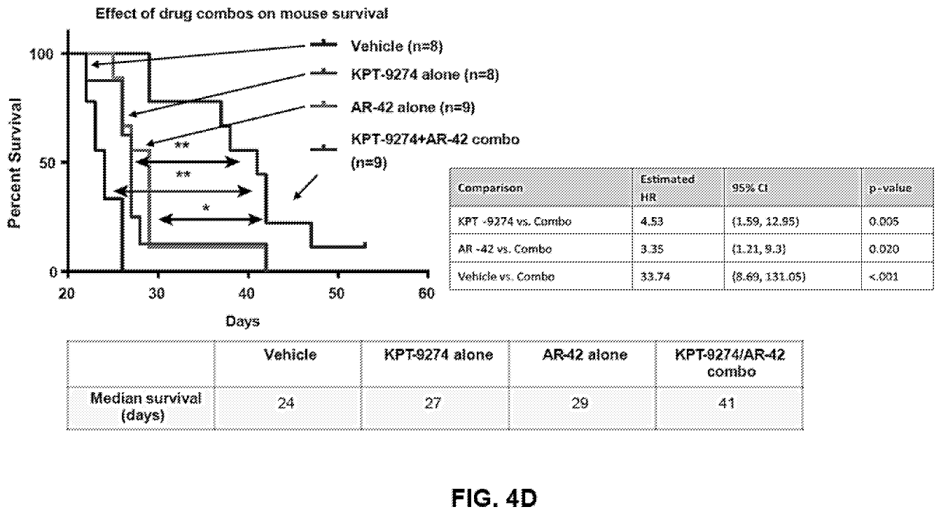

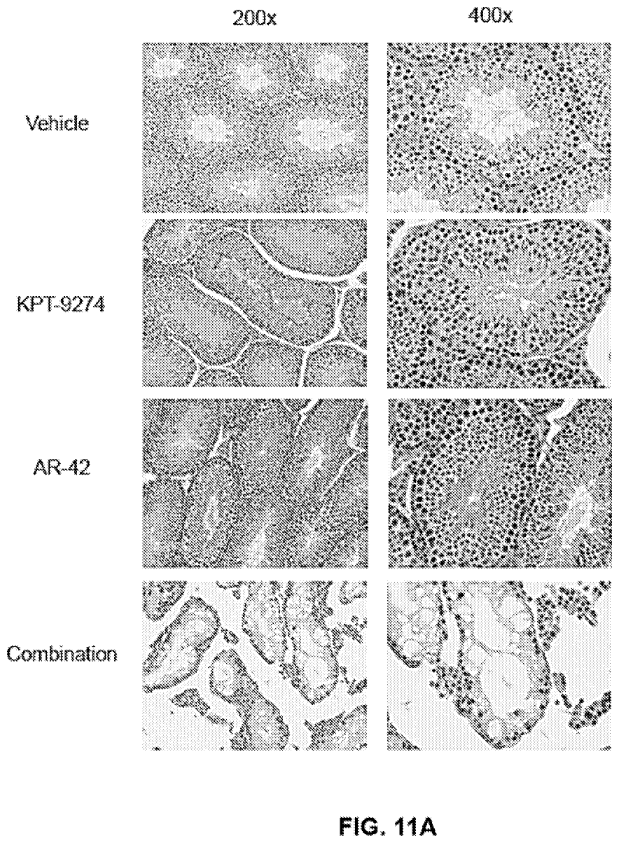

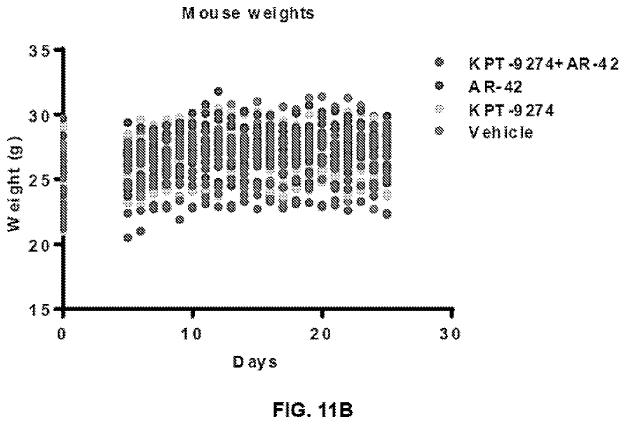

AR-42 enhances eradication of AML in vivo in combination with NAMPT inhibitor. To determine the clinical relevance of the drug combination, the efficacy of co-treatment of AR-42 and KPT-9274 was tested in the MOLM13 xenograft model. Briefly, 1×104 luciferase-transfected MOLM13 cells were transplanted by tail vein injection into NCG mice (FIG. 4A). Five days after engraftment, mice were randomized to receive vehicle, KPT-9274 alone (100 mg/kg daily), AR-42 alone (20 mg/kg every another day) or combination regime. The 20 mg/kg dose of AR-42 was tolerated according to an earlier pharmacokinetics study (Ref. 31). Based on IVIS bioluminescence imaging, the disease burden was lower in the drug combination group than in either single agent group or vehicle group over the course of the experiment (FIG. 4B). Histopathology of a variety of organs, including bone marrow, liver, lymph node and spleen, revealed that animals in combination groups have the least infiltrating neoplastic blasts and most differentiated hematopoietic cells (myeloid, erythroid, and megakaryocytic lineages) (FIG. 4C). Mice in the combination treatment group had vacuolation of the testes with atrophy of the seminiferous tubules and a lack of spermatogenesis (FIG. 11A). No overlapping toxicities between AR-42 and KPT-9274, like cytopenia, kidney injury and liver damage, were evident (FIG. 4C). In addition, we did not observe any noticeable weight loss in the AR-42 and drug combination groups over the course of the study (FIG. 11B).

Survival of vehicle-treated mice began to decline by week 3 (FIG. 4D). KPT-9274 or AR-42 as monotherapy added little to this effect, whereas mice in drug combination arm had a significantly longer survival. Specifically, mice in the KPT-9274 and AR-42 monotherapy groups had a median survival times of 27 and 29 days, respectively. Combined therapy prolonged the lifespan, conferring survival benefits on these MOLM13-engrafted mice (median survival time: 41 days).

The synthetic lethality of AR-42 and KPT-9274 is conferred by simultaneous suppression of HR and NHEJ pathways in leukemia initiating cells (LICs). AML relapse is driven by the inability of chemotherapy to eradicate leukemia stem/initiating cells. Since combined treatment of AR-42 and KPT-9274 abolishes the self-renewal potentials of patient LICs (FIG. 3D), the transcriptomes of LICs could be modulated by the drug treatments. To gain insights into the mechanism by which inhibition of HDAC overcomes KPT-9274 resistance, RNA-seq on patient LICs was performed. Bone marrow cells from 5 patients were treated with vehicle, single agents or drug combination ex vivo for short time period (12 hours). Treated LICs were analyzed for transcriptional differences and DNA repair mechanisms were highlighted.

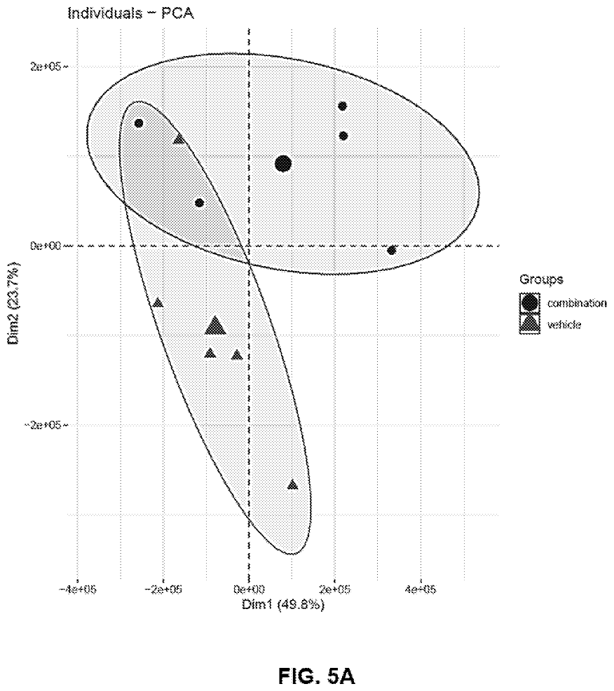

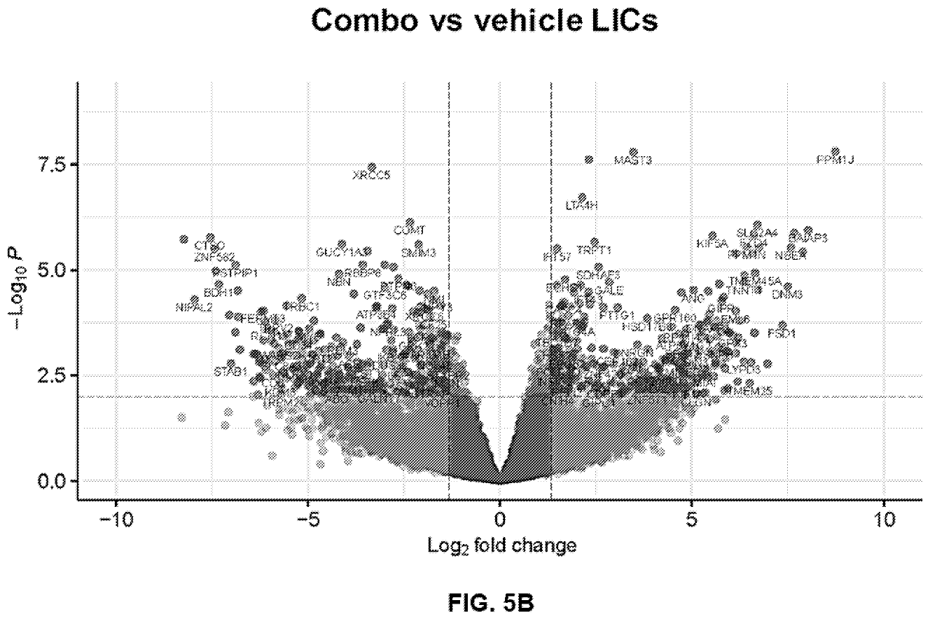

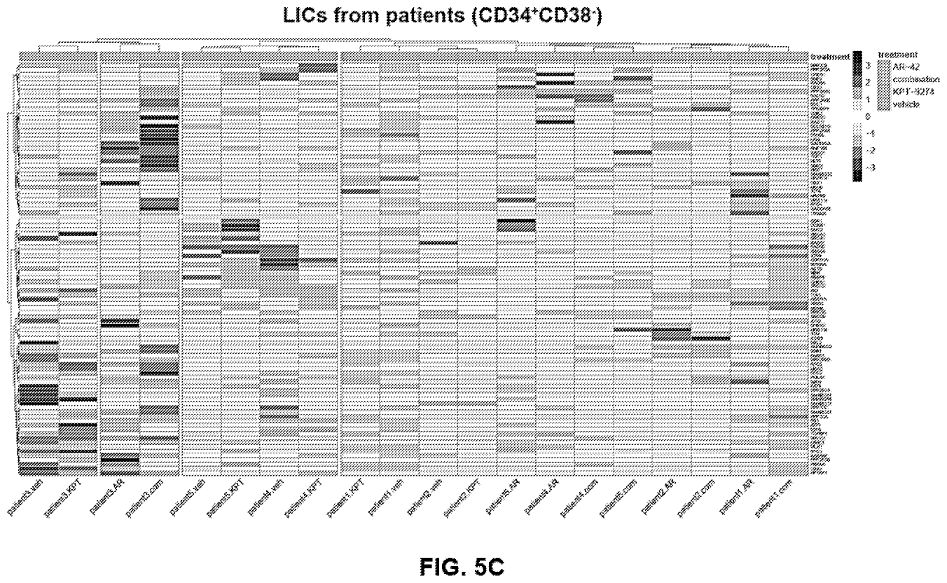

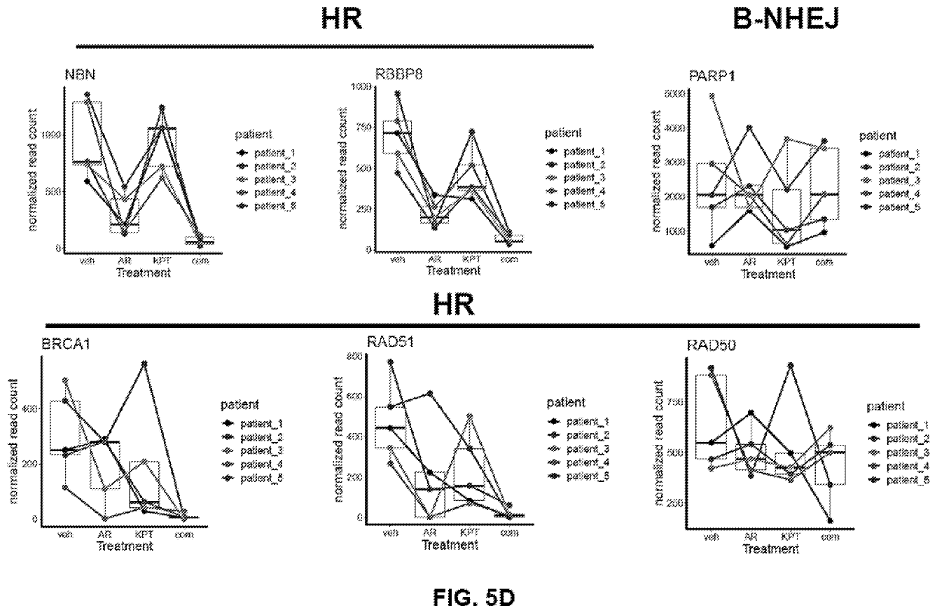

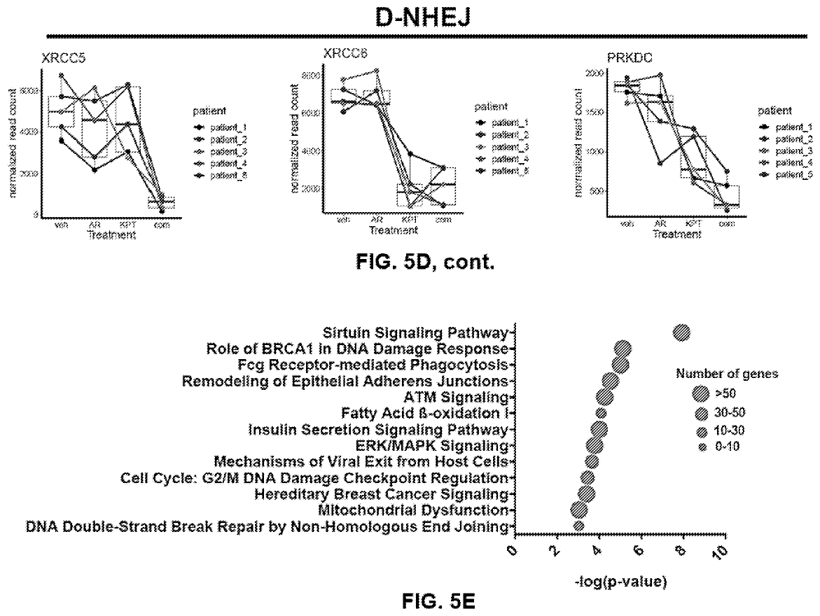

Although the transcriptomes from 5 patients were quite heterogenous, combination therapy-treated LICs are clustered separately from vehicle-treated populations in PCA analysis (FIG. 5A). Consistent with PCA analysis, the divergent responses of patient LICs to drug combination treatments were observed for transcriptome patterns and differentially expressed genes (DEGs). As displayed by volcano plot, genes promoting DNA repairs, like XRCC5, XRCC6, NBN and RBBP8, were differentially downregulated, while genes involved in transferase activity (MAST3) and serine/threonine phosphatase (PPMIJ) were differentially upregulated (FIG. 5B). Hierarchical clustering and heatmap analysis revealed that combined treatment induced a transcriptional response profile of HR and NHEJ pathways closely related to that of AR-42 or KPT-9274 treatment but distinct from that of vehicle treatment in LIC compartments (FIG. 5C).

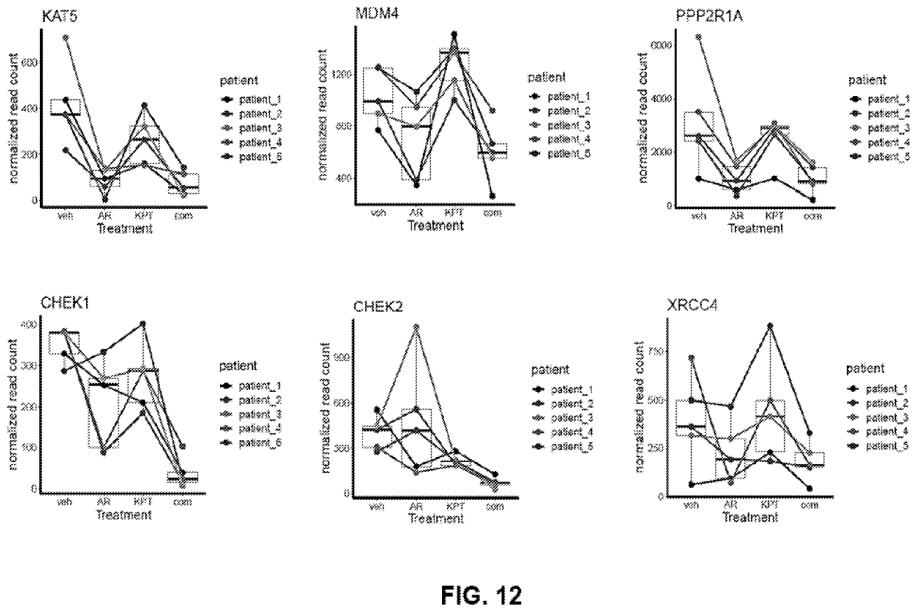

AR-42 alone or in combination with KPT-9274 downregulated the mRNA levels of HR genes, like NBN, RBBP8, RAD51 and BRCA1, D-NHEJ genes, like XRCC4 and XRCC5, and genes in ATM pathway (CHEK1 and CHEK2) (FIG. 5D; FIG. 12). Interestingly, BRCA1 and XRCC4 were upregulated by KPT-9274 alone in some patients. KPT-2974 treatment decreased the yields of D-NHEJ transcripts, like XRCC6 and PRKDC, and B-NHEJ gene, PARP1, in all patients. Based on these observations, gene set enrichment analysis on drug combination-treated samples relative to control was performed. Among the significantly enriched pathways were “The roles of BRCA1 in DNA damage response” and “ATM signaling”, suggestive of a potential impact of the combination therapy on DDR (FIG. 5E). Taken together, AR-42 and KPT-9274 synergistically impair HR, NHEJ and ATM pathways by abolishing gene transcription. Without wishing to be bound by a particular theory, this suggests that synthetic lethality may be due to ineffective DNA damage response.

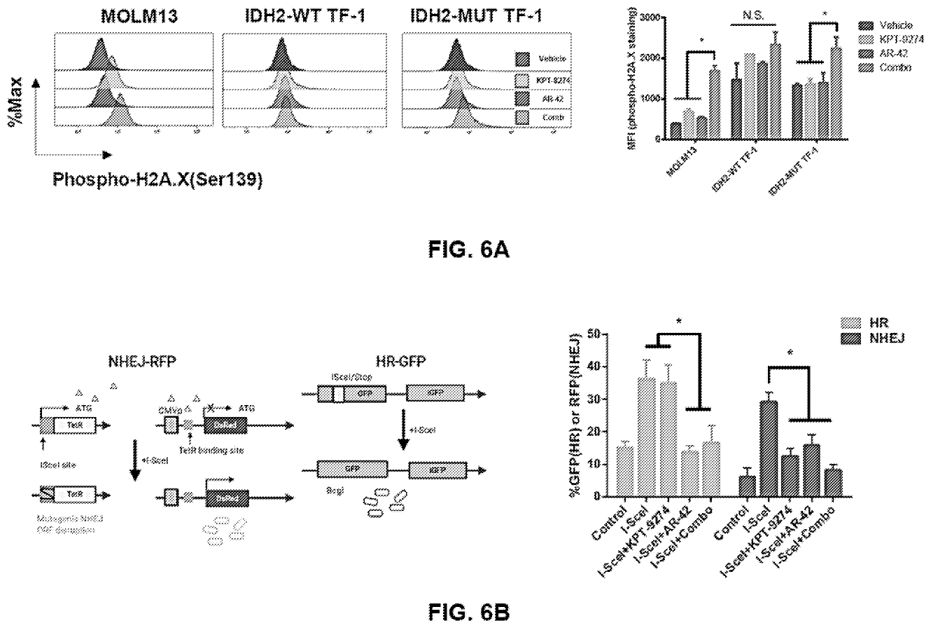

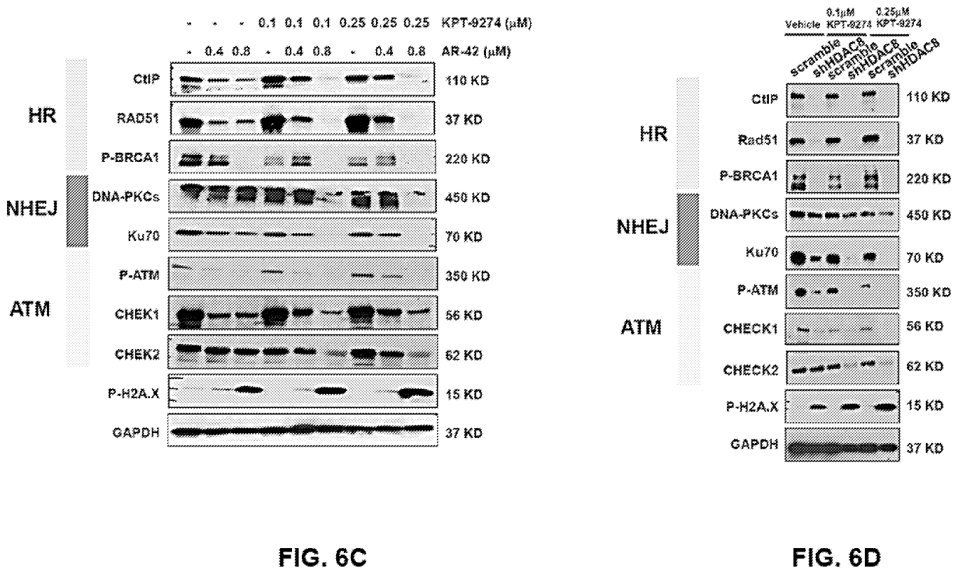

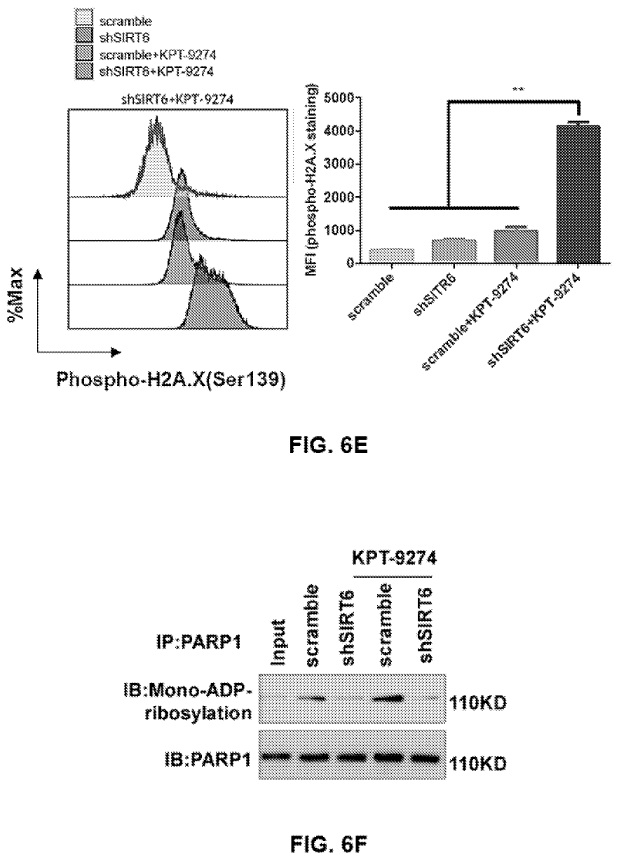

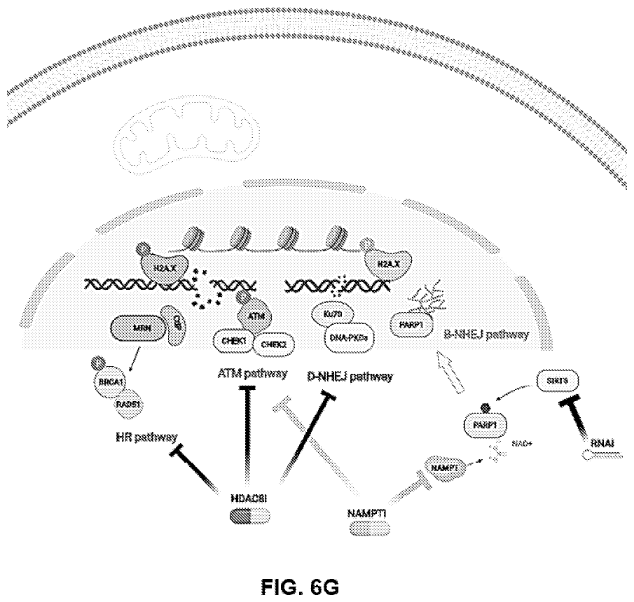

HDAC8 inhibition and SIRT6 knockdown cause the accumulation of DNA damage in KPT-9274-treated AML by impairing HR and D-NHEJ gene expressions and attenuating mono-ADP-ribosylation of PARP1. To further explore the mechanical basis of synthetic lethality of AR-42 and KPT-9274, intracellular phospho-H2A.X levels in AML cells which were exposed to vehicle, single agents, or drug combination for 48 hours were assessed. The drug combination created significantly more unrepaired DSBs as indicated by an increase in mean fluorescence intensity of phospho-H2A.X staining than either AR-42 or KPT-9274 alone in MOLM13 cells (FIG. 6A). Although AR-42 or KPT-9274 slightly increased fluorescence intensity of phospho-H2A.X staining in IDH2-WT TF-1 cells, the drug combination failed to further upregulate phospho-H2A.X levels. In contrast, the drug combination enhanced phospho-H2A.X staining in comparison with singe agents in IDH2-mutant TF-1 cells. Without wishing to be bound by a particular theory, the data suggest that IDH2 mutation synergizes with HDAC/NAMPT inhibition to impair DDR system. This effect might be attributed to HR deficiency caused by IDH2 mutation which may render cells more vulnerable to combined treatment.

It was unclear whether the potent ability of HDAC inhibitor to induce apoptosis of AML cells in combination with NAMPT inhibition was due to the loss of HR or NHEJ activity alone or both together. Accordingly, HR and NHEJ activities in AML cells exposed to these treatments were examined using the EJDR reporter system (FIG. 6B). EJDR-reporter-stable U2OS cells were treated with vehicle, single agents, or drug combination before being incubated with ligands, TA and Shield 1. KPT-9274 treatment did not impact HR activity as measured by HR-GFP reporter assay, whereas AR-42 treatment nearly completely abolished ISceI-induced HR. AR-42 treatment was also associated with NHEJ deficiency as measured by the reduction of the percentage of DsRed+ cells in the total population, but residual NHEJ activity was consistently detectable in AR-42-treated cells. KPT-9274 treatment caused a more robust reduction of NHEJ activity when applied alone or in combination with AR-42. Consistent with these findings, immunoblotting further revealed that 0.4 μM and 0.8 μM AR-42 treatment significantly reduced the levels of HR components, CtIP, Rad51, and phosphorylated BRCA1 as well as phosphorylated ATM as a monotherapy or in combination with KPT-9274 in MOLM13 cells (FIG. 6C). The expression of D-NHEJ pathway factors, Ku70 and DNA-PKcs, and ATM pathway mediators, CHEK1 and CHEK2, were abolished by AR-42 and KPT-9274 co-treatment. This effect was accompanied by induction of phospho-H2A.X. In agreement with pharmacological inhibition, knockdown of HDAC8 markedly reduced the levels of HR mediators, CtIP, Rad51, p-BRCA1, while shHDAC8 and KPT-9274 synergistically downregulated the abundance of D-NHEJ and ATM pathway mediators (FIG. 6D). Therefore, suppression of HDAC8 can attenuate HR and D-NHEJ gene expressions, resulting in NAMPT inhibitor sensitivity.

To elucidate the mechanism of the synergism between shSIRT6 and KPT-9274, the accumulation of phosph-H2A.X was measured. In the presence of KPT-9274, SIRT6-depleted cells exhibited more severe DNA damage as evidenced by increased phospho-H2A.X staining compared with scrambled control (FIG. 6E). KPT-9274 enhanced mono-ADP-ribosylation of PARP1 which was eradicated by knockdown of SIRT6, suggesting that SIRT6 was responsible for PARP1 mono-ADP-ribosylation (FIG. 6F). This observation provides a basis for further exploring the DDR-targeting mechanism of synthetic lethal effect exerted by simultaneous inhibition of NAMPT and HDAC8 or SIRT6 in AML.

### 4. Discussion

The present examples provide data development of synthetic lethal therapies that allow for lowering dosage of NAMPT inhibitors. The data show that that depletion of HDAC8 and SIRT6 conferred sensitivity to KPT-9274 treatment. Co-treatment with AR-42 and KPT-9274 resulted in a dramatic reduction of AML cell line and patient cell viability. Additionally, combination therapy showed superior efficacy towards MOLM13 xenograft and PDX models. Strikingly, AR-42/KPT-9274 combination abrogated the self-renewal of patient LICs by shutting down HR and NHEJ pathways. HDAC8i, shSIRT6 and NAMPTi simultaneously suppress compensatory DSB repair processes through the regulation of transcription and post-translational modifications (FIG. 6G). As inhibition of histone deacetylases and associated DNA repair machinery as a strategy to enhance NAMPT inhibitor efficacy has not been previously reported, the disclosed combination strategy represents a new clinically relevant approach to improve efficacy and tolerability of KPT-9274.

KPT-9274 is a dual PAK4/NAMPT inhibitor, allosterically binding to PAK4 and attenuating β-catenin as well as Wnt/β-catenin targets such as cyclin D1 and c-Myc. However, PAK4 is unlikely to play roles in the synergy mechanisms demonstrated herein as it has been previously shown that perturbance of PAK4 in AML does not alter the sensitivity of AML to KPT-9274 treatment (Ref. 5). Due to the lack of potent HDAC8-specific inhibitor, AR-42 was employed in the present study for in vivo and in vitro target validation. Although AR-42 targets multiple HDAC isoforms, the synthetic lethal effect with KPT-9274 observed is most likely to be attributed to its activity towards HDAC8, given HDAC8 was the highest ranked hit among all HDACs (the only one with p<0.01 in CRISPR screen data disclosed herein). Additionally, knockout of other major AR-42 targets, HDAC1, HDAC3 or HDAC6, did not display any synergy with KPT-9274. Simultaneous AR-42 and KPT-9274 treatment resulted in the accumulation of phospho-H2A.X and lethal DSBs. HDAC inhibitors disable functional HR by controlling the activities and expressions of HR-related genes. It was reported that the HDAC inhibitor vorinostat downregulates Rad50 and MRE11 protein levels in prostate cancer and lung adenocarcinoma cells (Ref. 32). In addition, inhibition of HDACs with PCI-2481 resulted in the reduction of Rad51 expression to weaken HR repair (Ref. 33). The roles of HDAC8 in HR were reported, as HDAC8 is associated with Rad51 and MRE11a and HDAC8 depletion leads to a decrease in Rad51 levels in multiple myeloma (Ref. 16). Consistent with these findings, the data disclosed herein detects substantial changes in the abundance of transcripts in HR pathway, like NBN and RBBP8(CtIP), in patient LICs upon AR-42 treatment (FIGS. 5C-D). A remarkable reduction was observed in the present data of transcripts in D-NHEJ and ATM signaling with cooperative actions of AR-42 and KPT-9274. The disclosed immunoblotting results on cell lines echo patient transcriptome data by showing that AR-42 or shHDAC8 primarily abrogated the expression of HR genes. At the same time, KPT-9274 and HDAC8 inhibition synergistically downregulated the components involved in D-NHEJ and ATM pathways at protein levels, sustaining DSBs. The present findings provide evidence that HDAC8i treatment suppresses HR and D-NHEJ (“BRCAness/DNA-PKness” phenotype), which, in combination with NAMPTi, causes synthetic lethality in AML cells due to accumulation of lethal DSBs beyond the reparable threshold.

IDH1/2 mutations exacerbate the HR deficiency by upregulating 2HG production. This “BRCAness” phenotype of IDH-mutant cells renders tumor exquisitely sensitive to PARP1 inhibitors (Ref. 30). In the current study, the treatment of IDH2-deficientAML cell lines or IDH1-deficient primary patient cells with KPT-9274 and AR-42 resulted in elevated accumulation of γH2A.X and greater inhibition of cell growth in comparison with treatment with individual inhibitors, while IDH-WT counterparts were mildly affected by these treatments. Without wishing to be bound by a particular theory, the mechanism of increased sensitivity of IDH1/2-mutant cells to AR-42 and KPT-9274 treatment may be due to the IDH1/2 mutation inducing deficiency in some compensatory HR pathways which cooperates with AR-42-induced HR and KPT-9274-induced NHEJ deficiencies to induce synergistic cell apoptosis. It was previously reported that 2HG accumulation induced by IDH1(R132) mutation in AML inhibits the function of histone demethylases (KDM4A and KDM4B) that are critical for HR function and consequently TIP60 and ATM activities are also decreased (Refs. 34-35). This effect is may be independent of HDAC inhibitor-mediated suppression of CtIP and BRCA1 functions. This reasoning provides a plausible explanation for why IDH1/2-mutant cells are more sensitive to NHEJ and HR inhibition than IDH1/2-WT cells. It is conceivable that inhibition of mutant IDH may attenuate 2-HG-mediated DNA repair defects and rescue the vulnerability of AML to drug combo treatment Therefore, it is rational to expect that co-treatment of AR-42 and KPT-9274 may benefit patients with mutant IDH1/2 in a 2-HG-dependent manner in clinical settings. It is noteworthy that IDH2-mutant TF-1 cells carry homozygous mutation of c.419G>A allele which is extremely rare occurrence in patients. Therefore, IDH2 could be used as a precision medicine marker for identifying AML patients that may benefit from a therapeutic regime combining NAMPTi and HDACi.

LICs are associated with relapse and drug resistance (36,37). Compared with differentiated tumor, LICs are particularly tolerant to DNA damage due to p21-dependent cell cycle arrest (Ref. 39). They are addicted to error-prone NHEJ for repairing oxidative stress-induce damage (Ref. 39). Pharmacological inhibition of CHEK1 was shown to enhance HDAC inhibitor activity toward LICs by disrupting HR repair (Ref. 40). The examples disclosed herein demonstrate that AR-42 and KPT-2974 cooperate to shut down multiple DDR pathways and decreased self-renewal of LICs but not normal hematopoietic stem cells. AR-42 was previously reported to induce death of leukemia stem cells by triggering caspase-dependent apoptosis (Ref. 28). However, the examples herein demonstrate for the first time that AR-42 can eradicate LICs in a combination therapy at sublethal doses. By probing LIC transcriptomes, it was found that a strong DDR-suppressive effect was achieved through combined treatments, while LICs treated with KPT-9274 alone augmented the levels of HR genes in some patients. This raises the possibility that KPT-9274-treated LIC populations could be particularly vulnerable to this anti-HR strategy. This is supported by the observation that inhibition of NAMPT downstream molecule, PARP1, induces accumulation of Rad51 and preserves HR responses in ovarian cancer (Ref. 41).

Prior studies showed that there exists a poor-prognostic subset of AML patients, with widespread genomic instability, relying on SIRT6 and NHEJ to compensate for DNA-replication stress (Ref. 1 and 42). Silencing of SIRT6 leads to impaired recruitment of BRCA1 and 53BP1, which are involved in HR (Ref. 43) Another study suggests that SIRT6 inhibition compromises the ability of leukemia cells to repair DSBs that, in turn, increases their sensitivity to daunorubicin and Ara-C(Ref. 42). SIRT6 is known to stimulate NHEJ in the absence of DNA-PKcs, the D-NHEJ enzyme. Independent of its deacetylase function, SIRT6 mono-ADP ribosylates, PARP1, at lysine 521, thereby stimulating its poly-ADP ribosylation activity (Refs. 13 and 44). This modification was required for SIRT6-mediated stimulation of DSB repair, cooperating with NAMPT to fully unlock PARP1-mediated B-NHEJ. The present disclosure provides a rationale of developing therapies targeting mono-ADP-ribosylation activity of SIRT6 while sparing deacetylase function to minimize toxicity for healthy cells.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A pharmaceutical composition comprising a pharmaceutically acceptable carrier and a therapeutically effective amount of: (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor, or a pharmaceutically acceptable salt, hydrate, solvate, or polymorph thereof.

2. A method for the treatment of AML in a mammal comprising the step of administering to the mammal a therapeutically effective amount of: (a) at least one disclosed NAMPT inhibitor, or a pharmaceutically acceptable salt, hydrate, solvate, or polymorph thereof; and (b) at least one HDAC8 inhibitor and/or at least one SIRT6 inhibitor, or a pharmaceutically acceptable salt, hydrate, solvate, or polymorph thereof.

3. The method of claim 2, wherein the administering comprises administering the pharmaceutical composition of claim 1.

4. The method of claim 2, wherein the administering is co-administering the at least one disclosed NAMPT inhibitor and the at least one HDAC8 inhibitor and/or at least one SIRT6 inhibitor.

5. The method of claim 4, wherein the co-administering is simultaneous administration of the at least one disclosed NAMPT inhibitor and the at least one HDAC8 inhibitor and/or at least one SIRT6 inhibitor.

6. The method of claim 5, wherein the simultaneous administration comprises co-administering the at least one disclosed NAMPT inhibitor and the at least one HDAC8 inhibitor and/or at least one SIRT6 inhibitor.

7. The method of claim 5, wherein the simultaneous administration comprises co-administering a first pharmaceutical composition comprising the at least one disclosed NAMPT inhibitor and a second pharmaceutical composition comprising the at least one HDAC8 inhibitor and/or at least one SIRT6 inhibitor.

8. The method of claim 5, wherein simultaneous administration comprises co-administering a pharmaceutical composition of claim 1.

9. The method of claim 4, wherein the co-administrating is sequential administration of the at least one disclosed NAMPT inhibitor and the at least one HDAC8 inhibitor and/or at least one SIRT6 inhibitor.

10. The method of claim 9, wherein the sequential administration comprises administering a first pharmaceutical composition comprising the at least one disclosed NAMPT inhibitor and followed by administering a second pharmaceutical composition comprising the at least one HDAC8 inhibitor and/or at least one SIRT6 inhibitor.
